(19) United States
(12) Patent Application Publication
(10) Pub. No.: US 2006/0140963 A1
(43) Pub. Date: Jun. 29, 2006

(54) Cytotoxicity mediation of cells evidencing surface expression of CD59

(75) Inventors: David S .F. Young, Toronto (CA); Helen P. Findlay, Toronto (CA); Susan E. Hahn, Toronto (CA); Lisa M. Cechetto, Ancaster (CA); Luis A. G. da Cruz, Toronto (CA)

Correspondence Address: MCHALE & SLAVIN, P.A., 2855 PGA BLVD, PALM BEACH GARDENS, FL, 33410, US

(73) Assignee: Arius Research, Inc.

(21) Appl. No.: 11/361,153

(22) Filed: Feb. 24, 2006

Related U.S. Application Data
(63) Continuation-in-part of application No. 10/944,664, filed on Sep. 15, 2004.
Application No. 10/944,664 is a continuation-in-part of application No. 10/413,755, filed on Apr. 14, 2003, now Pat. No. 6,794,494.

(51) Int. Cl. G01N 33/574; A61K 39/395; C07K 16/30

(52) U.S. Cl. 424/155.1; 435/7.23; 530/388.8

(57) ABSTRACT

This invention relates to the diagnosis and treatment of cancerous diseases, particularly to the mediation of cytotoxicity of tumor cells; and most particularly to the use of cancerous disease modifying antibodies (CDMAB), optionally in combination with one or more chemotherapeutic agents, as a means for initiating the cytotoxic response. The invention further relates to binding assays which utilize the CDMAB of the instant invention.

### REFERENCE TO RELATED APPLICATIONS

This application is a continuation-in-part to U.S. patent application Ser. No. 10/944,664 filed Sep. 15, 2004 which is a continuation-in-part to U.S. patent application Ser. No. 10/413,755, filed Apr. 14, 2003, now U.S. Pat. No. 6,794,494, and is a continuation-in-part to U.S. patent application Ser. No. 11/067,366, filed Feb. 25, 2005, which relies upon U.S. Provisional Application No. 60/548,667, filed Feb. 26, 2004, the contents of each of which are herein incorporated by reference.

### FIELD OF THE INVENTION

This invention relates to the diagnosis and treatment of cancerous diseases, particularly to the mediation of cytotoxicity of tumor cells; and most particularly to the use of cancerous disease modifying antibodies (CDMAB), optionally in combination with one or more chemotherapeutic agents, as a means for initiating the cytotoxic response. The invention further relates to binding assays, which utilize the CDMAB of the instant invention

### BACKGROUND OF THE INVENTION

CD59 is an 18-20 kDa glycosyl phosphatidylinositol (GPI)-anchored membrane glycoprotein. It was initially isolated from the surface of human erythrocytes, and functions as an inhibitor of complement activation. Several antibodies that were developed to enhance complement-mediated lysis were subsequently found to target CD59. Their independent development led to the multitude of names by for CD59, including MEM-43 antigen, membrane inhibitor of reactive lysis (MIRL), H19, membrane attack complex-inhibitory factor (MACIF), homologous restriction factor with m.w. 20,000 (HRF20) and protectin (Walsh, Tone et al. 1992).

The CD59 antigen has been well characterized by amino acid analysis and NMR. It consists of 128 amino acids, of which the first 25 comprise a signal sequence. There are 10 cysteine residues, which result in a tightly folded molecule. The asparagine residue at position 18 is known to be N-glycosylated, while the asparagine residue at position 77 is linked to the GPI anchor. The C-terminus residues are characteristic of GPI-anchored proteins (Davies and Lachmann 1993).

CD59 was initially discovered on the surface of human erythrocytes, but is a widely expressed molecule. A large collection of data on cellular distribution from flow cytometry, immunohistochemistry and Northern blot analysis has revealed expression on many types of cells and tissues, including hematopoietic cells such as, platelets, leukocytes and fibroblasts, as well as erythrocytes (Meri, Waldmann et al. 1991). CD59 is abundant on vascular and ductal endothelium throughout the body, particularly in kidneys, bronchus, pancreas, skin epidermis and biliary and salivary glands (Meri, Waldmann et al. 1991). Expression has been noted in the lung, liver, placenta, thyroid and spermatozoa (Davies and Lachmann 1993). Soluble forms of CD59 have been detected in saliva, urine, tears, sweat, cerebrospinal fluid, breast milk, amniotic fluid and seminal plasma (Davies and Lachmann 1993). The origin of soluble CD59 has yet to be determined; whether it is secreted, cleaved by phospholipases or shed from cells by other means remains unknown (Davies and Lachmann 1993). CD59 appears to be absent from many B cell lines, CNS tissue, liver parenchyma and pancreatic Islets of Langerhans (Meri, Waldmann et al. 1991).

Although CD59 is widely expressed in normal cells and tissues, it is also widely expressed on malignant tumors. There is evidence that the expression of CD59 is increased compared to normal tissue in certain types of cancer, and that the level of expression correlates with the stage of differentiation of the tumor. Moderate to high levels of CD59 expression have been reported in thyroid, prostate, breast, ovarian, lung, colorectal, pancreatic, gastric, renal and skin cancers as well as in malignant glioma, leukemia and lymphoma (Fishelson, Donin et al. 2003).

CD59 is known to inhibit the formation of the membrane attack complex (MAC) following complement activation. MAC formation is one of the final events in the complement cascade, forming a pore in the cellular membrane, which ultimately leads to the destruction of the cell. CD59 binds to C5b-8 and interferes with the subsequent polymerization of C9 molecules and MAC formation. Other complement inhibitory proteins such as complement receptor type-1 (CR1; CD35), membrane cofactor protein (MCP; CD46) and decay accelerating factor (DAF; CD55) act earlier on in the complement cascade. Complement activation results in either destruction of the targeted cell or to cell activation, which recruits leukocytes, contracts surrounding smooth muscle and increases vascular permeability. Complement activation also plays a role in antibody-dependent cellular cytotoxicity (ADCC) and complement-dependent cellular cytotoxicity (CDCC). Complement activation results in an inflammatory response that could damage targeted tissues if poorly regulated. CD59 and other complement inhibitory proteins prevent autologous tissue damage from activation of the complement cascade. It has been postulated that over-expression of complement inhibitory proteins such as CD59 may contribute to enhanced resistance to complement activation that malignant tumors often acquire (Jarvis, Li et al. 1997). If this is the case, treatment with monoclonal antibodies directed against complement inhibitory proteins could overcome this resistance, making the tumor more responsive to immunotherapy or other treatments.

Paroxysmal nocturnal hemoglobinuria (PNH) is a rare heritable disorder that affects hematopoietic stem cells, resulting in cells that are abnormally sensitized to complement attack (Davies and Lachmann 1993). The symptoms include chronic hemolysis, anemia and thrombosis (Sugita and Masuho 1995). Cells affected by PNH, including erythrocytes, granulocytes, monocytes, platelets and sometimes lymphocytes, are deficient in GPI-anchored proteins (Davies and Lachmann 1993). Affected cells lack acetylcholinesterase, LFA-3, HUPAR and complement regulator proteins CD35, CD46, CD55 and CD59 (Davies and Lachmann 1993). There is a single reported case of an individual that is completely lacking expression of CD59 but none of the other complement regulatory GPI-anchored proteins. This deficiency is associated with PNH-like symptoms such as hemolytic anemia and thrombosis (Davies and Lachmann 1993). Although there are undesirable effects associated with lack of CD59 function, this individual proves that complete loss is non-lethal. Hemolytic side effects are an acceptable obstacle to overcome when faced with the daunting task of treating cancer.

A mouse model in which one of the CD59 genes have been knocked out has also demonstrated that CD59 deficiency is non-lethal in vivo. Mice express two forms of CD59, CD59a and CD59b. CD59a is widely expressed in various mouse tissues including blood cells, whereas CD59b expression has only been identified in the testis. Miwa et al. generated CD59a-deficient mice in order to assess the role of CD59 to protect erythrocytes from spontaneous complement attack in vivo. They reported that the knockout mice developed and lived normally without any signs of hemolytic anemia, and that hemoglobin levels were not significantly elevated. Despite erythrocytes being more sensitive to induced complement attack by injection with cobra venom factor (CVF), erythrocyte elimination from spontaneous complement attack was not significantly elevated as compared to wild type (Miwa, Zhou et al. 2002).

A 21-kDa membrane glycoprotein called rat inhibitory protein (RIP) has been identified in rats. RIP inhibits MAC assembly at or after C5b-8 stage and is released from rat erythrocytes by phosphatidylinositol-specific phospholipase C. These factors, along with the N-terminal sequence, suggest that RIP is the rat homologue of human CD59. F(ab′)₂ fragments of 6D1, a mouse monoclonal antibody directed against rat RIP, was administered to a group of male Wistar rats. In the same study, fragments of 512, an antibody directed against a different rat membrane-associated complement regulatory protein, were also administered. There was no change observed in heart rate or blood pressure following injection of 6D1 fragments. Fragment binding was detected in lung, heart and liver. The only observed effects were a small increase in leukocyte count and decrease in erythrocyte count; there was no change in number of platelets. In contrast, injection with 512 fragments resulted in a rapid increase in blood pressure, a rapid decrease in leukocytes and platelets, and a continuously increasing erythrocyte count up to 2 hours following injection (Matsuo, Ichida et al. 1994).

The chimeric monoclonal antibody Rituximab (Rituxan, Genentech, San Francisco, Calif.) is directed against the CD20 antigen, and has been approved for use in treatment of non-Hodgkin's lymphoma (NHL). Many patients that are CD20⁺ are unresponsive to treatment, and most patients who do respond will eventually develop resistance to treatment. In an effort to overcome this resistance, use of anti-CD59 antibodies to increase CDCC has been investigated. NHL and MM cell lines that are resistant to Rituxan treatment in the presence of complement in vitro express CD59, whereas NHL and MM cell lines that are sensitive to the same treatment do no express CD59. Pre-incubation of one of the resistant cell lines with an anti-CD59 antibody (YTH53.1) sensitized the cells to treatment with Rituximab and human complement. High expression levels of CD59 have also been exhibited on tumors isolated from patients that are CD20⁺ but have had disease progression with Rituximab treatment (Treon, Emmanouilides et al. 2005).

The activity of the CD59 antibody YTH53.1 in vitro has been evaluated on breast cancer (T47D) and ovarian teratocarcinoma (PA-1) using three-dimensional microtumor spheroids (MTS). MTS are multicellular aggregates that grow in culture and represent a model closer to that observed in vivo than monolayer or suspension cultures. Previous work by this group had shown that PA-1 cells grown as MTS were more resistant to complement lysis than PA-1 cells grown in suspension. To evaluate whether this resistance could be overcome, cytotoxicity was measured by chromium release assay and cell damage was visualized by uptake of propidium iodide (PI) following pre-treatment of MTS with biotinylated YTH53.1. The antibody retained its affinity for CD59 with biotinylation but lost its capacity to activate the classical complement pathway. Rabbit anti-human polyclonal antibody raised against breast cancer cells (S2) was used to activate the classical pathway. Overnight incubation with YTH53.1 led to total infiltration of the MTS, and the chromium release assay showed 33% of cells were killed after a 1 to 2-hour lag phase in the presence of YTH53.1, S2 and human complement. Electron microscopy revealed the average T47D tumor volume decreased 28% following incubation with YTH53.1, S2 and human complement. Fluorescence microscopy following PI incubation revealed several layers of cell death on T47D and PA-1 MTS following incubation with YTH53.1, S2 and human complement. These results combined indicate that an anti-CD59 antibody can increase the complement-mediated lysis of tumor cells in vitro (Hakulinen and Meri 1998).

Another group found that the resistance to complement-mediated lysis of the human metastatic prostate adenocarcinoma cell lines DU145 and PC3 could be overcome in vitro by treating with the CD59 antibody YTH53.1. The chromium release assay was used to measure cell death in the presence and absence of YTH53.1 and biotinylated-YTH53.1. Without the CD59 antibodies, both cell lines were completely resistant to complement-mediated lysis. Treating with YTH53.1 partially overcame this resistance by killing 56% of PC3 cells and 34% of DU145 cells. Treatment with biotinylated-YTH53.1 overcame the resistance to a lesser extent; 47% of PC3 and 20% of DU145 cells were killed. The increased sensitivity of PC3 compared to DU145 can be attributed to the increased expression of CD59 by PC3. The differential effect of the native and biotinylated antibody demonstrates the combined effect of activation of the classical pathway of complement and the neutralization of CD59, as the biotinylated antibody presumably does not activate the classical pathway (Jarvis, Li et al. 1997). The bulk of the activity of the antibody may be attributed to the blocking of complement inhibition (neutralization of CD59), as adding complement activation by the classical pathway only increases activity a marginal amount (e.g. 47% for biotinylated-YTH53.1 versus 56% for YTH53.1 on PC3 cells) (Jarvis, Li et al. 1997). There has been no in vivo analysis of the anti-CD59 antibody YTH53.1 to date. There are no reports of any anti-CD59 antibodies exhibiting therapeutic efficacy in preclinical cancer models in vivo.

Monoclonal Antibodies as Cancer Therapy: Each individual who presents with cancer is unique and has a cancer that is as different from other cancers as that person's identity. Despite this, current therapy treats all patients with the same type of cancer, at the same stage, in the same way. At least 30% of these patients will fail the first line therapy, thus leading to further rounds of treatment and the increased probability of treatment failure, metastases, and ultimately, death. A superior approach to treatment would be the customization of therapy for the particular individual. The only current therapy which lends itself to customization is surgery. Chemotherapy and radiation treatment cannot be tailored to the patient, and surgery by itself, in most cases is inadequate for producing cures.

With the advent of monoclonal antibodies, the possibility of developing methods for customized therapy became more realistic since each antibody can be directed to a single epitope. Furthermore, it is possible to produce a combination of antibodies that are directed to the constellation of epitopes that uniquely define a particular individual's tumor.

Having recognized that a significant difference between cancerous and normal cells is that cancerous cells contain antigens that are specific to transformed cells, the scientific community has long held that monoclonal antibodies can be designed to specifically target transformed cells by binding specifically to these cancer antigens; thus giving rise to the belief that monoclonal antibodies can serve as “Magic Bullets” to eliminate cancer cells. However, it is now widely recognized that no single monoclonal antibody can serve in all instances of cancer, and that monoclonal antibodies can be deployed, as a class, as targeted cancer treatments. Monoclonal antibodies isolated in accordance with the teachings of the instantly disclosed invention have been shown to modify the cancerous disease process in a manner which is beneficial to the patient, for example by reducing the tumor burden, and will variously be referred to herein as cancerous disease modifying antibodies (CDMAB) or “anti-cancer” antibodies.

At the present time, the cancer patient usually has few options of treatment. The regimented approach to cancer therapy has produced improvements in global survival and morbidity rates. However, to the particular individual, these improved statistics do not necessarily correlate with an improvement in their personal situation.

Thus, if a methodology was put forth which enabled the practitioner to treat each tumor independently of other patients in the same cohort, this would permit the unique approach of tailoring therapy to just that one person. Such a course of therapy would, ideally, increase the rate of cures, and produce better outcomes, thereby satisfying a long-felt need.

Historically, the use of polyclonal antibodies has been used with limited success in the treatment of human cancers. Lymphomas and leukemias have been treated with human plasma, but there were few prolonged remission or responses. Furthermore, there was a lack of reproducibility and there was no additional benefit compared to chemotherapy. Solid tumors such as breast cancers, melanomas and renal cell carcinomas have also been treated with human blood, chimpanzee serum, human plasma and horse serum with correspondingly unpredictable and ineffective results.

There have been many clinical trials of monoclonal antibodies for solid tumors. In the 1980s there were at least four clinical trials for human breast cancer which produced only one responder from at least 47 patients using antibodies against specific antigens or based on tissue selectivity. It was not until 1998 that there was a successful clinical trial using a humanized anti-Her2/neu antibody (Herceptin®) in combination with Cisplatin. In this trial 37 patients were assessed for responses of which about a quarter had a partial response rate and an additional quarter had minor or stable disease progression. The median time to progression among the responders was 8.4 months with median response duration of 5.3 months.

Herceptin® was approved in 1998 for first line use in combination with Taxol®. Clinical study results showed an increase in the median time to disease progression for those who received antibody therapy plus Taxol® (6.9 months) in comparison to the group that received Taxol® alone (3.0 months). There was also a slight increase in median survival; 22 versus 18 months for the Herceptin® plus Taxol® treatment arm versus the Taxol® treatment alone arm. In addition, there was an increase in the number of both complete (8 versus 2 percent) and partial responders (34 versus 15 percent) in the antibody plus Taxol® combination group in comparison to Taxol® alone. However, treatment with Herceptin® and Taxol® led to a higher incidence of cardiotoxicity in comparison to Taxol® treatment alone (13 versus 1 percent respectively). Also, Herceptin® therapy was only effective for patients who over express (as determined through immunohistochemistry (IHC) analysis) the human epidermal growth factor receptor 2 (Her2/neu), a receptor, which currently has no known function or biologically important ligand; approximately 25 percent of patients who have metastatic breast cancer. Therefore, there is still a large unmet need for patients with breast cancer. Even those who can benefit from Herceptin® treatment would still require chemotherapy and consequently would still have to deal with, at least to some degree, the side effects of this kind of treatment.

The clinical trials investigating colorectal cancer involve antibodies against both glycoprotein and glycolipid targets. Antibodies such as 17-1A, which has some specificity for adenocarcinomas, has undergone Phase 2 clinical trials in over 60 patients with only 1 patient having a partial response. In other trials, use of 17-1A produced only 1 complete response and 2 minor responses among 52 patients in protocols using additional cyclophosphamide. To date, Phase III clinical trials of 17-1A have not demonstrated improved efficacy as adjuvant therapy for stage III colon cancer. The use of a humanized murine monoclonal antibody initially approved for imaging also did not produce tumor regression.

Only recently have there been any positive results from colorectal cancer clinical studies with the use of monoclonal antibodies. In 2004, ERBITUX® was approved for the second line treatment of patients with EGFR-expressing metastatic colorectal cancer who are refractory to irinotecan-based chemotherapy. Results from both a two-arm Phase II clinical study and a single arm study showed that ERBITUX® in combination with irinotecan had a response rate of 23 and 15 percent respectively with a median time to disease progression of 4.1 and 6.5 months respectively. Results from the same two-arm Phase II clinical study and another single arm study showed that treatment with ERBITUX® alone resulted in an 11 and 9 percent response rate respectively with a median time to disease progression of 1.5 and 4.2 months respectively.

Consequently in both Switzerland and the United States, ERBITUX® treatment in combination with irinotecan, and in the United States, ERBITUX® treatment alone, has been approved as a second line treatment of colon cancer patients who have failed first line irinotecan therapy. Therefore, like Herceptin®, treatment in Switzerland is only approved as a combination of monoclonal antibody and chemotherapy. In addition, treatment in both Switzerland and the US is only approved for patients as a second line therapy. Also, in 2004, AVASTIN® was approved for use in combination with intravenous 5-fluorouracil-based chemotherapy as a first line treatment of metastatic colorectal cancer. Phase III clinical study results demonstrated a prolongation in the median survival of patients treated with AVASTIN® plus 5-fluorouracil compared to patients treated with 5-fluourouracil alone (20 months versus 16 months respectively). However, again like Herceptin® and ERBITUX®, treatment is only approved as a combination of monoclonal antibody and chemotherapy.

There also continues to be poor results for lung, brain, ovarian, pancreatic, prostate, and stomach cancer. The most promising recent results for non-small cell lung cancer came from a Phase II clinical trial where treatment involved a monoclonal antibody (SGN-15; dox-BR96, anti-Sialyl-LeX) conjugated to the cell-killing drug doxorubicin in combination with the chemotherapeutic agent Taxotere. Taxotere is the only FDA approved chemotherapy for the second line treatment of lung cancer. Initial data indicate an improved overall survival compared to Taxotere alone. Out of the 62 patients who were recruited for the study, two-thirds received SGN-15 in combination with Taxotere while the remaining one-third received Taxotere alone. For the patients receiving SGN-15 in combination with Taxotere, median overall survival was 7.3 months in comparison to 5.9 months for patients receiving Taxotere alone. Overall survival at 1 year and 18 months was 29 and 18 percent respectively for patients receiving SNG-15 plus Taxotere compared to 24 and 8 percent respectively for patients receiving Taxotere alone. Further clinical trials are planned.

Preclinically, there has been some limited success in the use of monoclonal antibodies for melanoma. Very few of these antibodies have reached clinical trials and to date none have been approved or demonstrated favorable results in Phase III clinical trials.

The discovery of new drugs to treat disease is hindered by the lack of identification of relevant targets among the products of 30,000 known genes that unambiguously contribute to disease pathogenesis. In oncology research, potential drug targets are often selected simply due to the fact that they are over-expressed in tumor cells. Targets thus identified are then screened for interaction with a multitude of compounds. In the case of potential antibody therapies, these candidate compounds are usually derived from traditional methods of monoclonal antibody generation according to the fundamental principles laid down by Kohler and Milstein (1975, Nature, 256, 495-497, Kohler and Milstein). Spleen cells are collected from mice immunized with antigen (e.g. whole cells, cell fractions, purified antigen) and fused with immortalized hybridoma partners. The resulting hybridomas are screened and selected for secretion of antibodies which bind most avidly to the target. Many therapeutic and diagnostic antibodies directed against cancer cells, including Herceptin® and RITUXIMAB, have been produced using these methods and selected on the basis of their affinity. The flaws in this strategy are twofold. Firstly, the choice of appropriate targets for therapeutic or diagnostic antibody binding is limited by the paucity of knowledge surrounding tissue specific carcinogenic processes and the resulting simplistic methods, such as selection by overexpression, by which these targets are identified. Secondly, the assumption that the drug molecule that binds to the receptor with the greatest affinity usually has the highest probability for initiating or inhibiting a signal may not always be the case.

Despite some progress with the treatment of breast and colon cancer, the identification and development of efficacious antibody therapies, either as single agents or co-treatments, has been inadequate for all types of cancer.

Prior Patents:

U.S. Pat. No. 5,750,102 discloses a process wherein cells from a patient's tumor are transfected with MHC genes which may be cloned from cells or tissue from the patient. These transfected cells are then used to vaccinate the patient.

U.S. Pat. No. 4,861,581 discloses a process comprising the steps of obtaining monoclonal antibodies that are specific to an internal cellular component of neoplastic and normal cells of the mammal but not to external components, labeling the monoclonal antibody, contacting the labeled antibody with tissue of a mammal that has received therapy to kill neoplastic cells, and determining the effectiveness of therapy by measuring the binding of the labeled antibody to the internal cellular component of the degenerating neoplastic cells. In preparing antibodies directed to human intracellular antigens, the patentee recognizes that malignant cells represent a convenient source of such antigens.

U.S. Pat. No. 5,171,665 provides a novel antibody and method for its production. Specifically, the patent teaches formation of a monoclonal antibody which has the property of binding strongly to a protein antigen associated with human tumors, e.g. those of the colon and lung, while binding to normal cells to a much lesser degree.

U.S. Pat. No. 5,484,596 provides a method of cancer therapy comprising surgically removing tumor tissue from a human cancer patient, treating the tumor tissue to obtain tumor cells, irradiating the tumor cells to be viable but non-tumorigenic, and using these cells to prepare a vaccine for the patient capable of inhibiting recurrence of the primary tumor while simultaneously inhibiting metastases. The patent teaches the development of monoclonal antibodies which are reactive with surface antigens of tumor cells. As set forth at col. 4, lines 45 et seq., the patentees utilize autochthonous tumor cells in the development of monoclonal antibodies expressing active specific immunotherapy in human neoplasia.

U.S. Pat. No. 5,693,763 teaches a glycoprotein antigen characteristic of human carcinomas and not dependent upon the epithelial tissue of origin.

U.S. Pat. No. 5,783,186 is drawn to Anti-Her2 antibodies which induce apoptosis in Her2 expressing cells, hybridoma cell lines producing the antibodies, methods of treating cancer using the antibodies and pharmaceutical compositions including said antibodies.

U.S. Pat. No. 5,849,876 describes new hybridoma cell lines for the production of monoclonal antibodies to mucin antigens purified from tumor and non-tumor tissue sources.

U.S. Pat. No. 5,869,268 is drawn to a method for generating a human lymphocyte producing an antibody specific to a desired antigen, a method for producing a monoclonal antibody, as well as monoclonal antibodies produced by the method. The patent is particularly drawn to the production of an anti-HD human monoclonal antibody useful for the diagnosis and treatment of cancers.

U.S. Pat. No. 5,869,045 relates to antibodies, antibody fragments, antibody conjugates and single chain immunotoxins reactive with human carcinoma cells. The mechanism by which these antibodies function is two-fold, in that the molecules are reactive with cell membrane antigens present on the surface of human carcinomas, and further in that the antibodies have the ability to internalize within the carcinoma cells, subsequent to binding, making them especially useful for forming antibody-drug and antibody-toxin conjugates. In their unmodified form the antibodies also manifest cytotoxic properties at specific concentrations.

U.S. Pat. No. 5,780,033 discloses the use of autoantibodies for tumor therapy and prophylaxis. However, this antibody is an antinuclear autoantibody from an aged mammal. In this case, the autoantibody is said to be one type of natural antibody found in the immune system. Because the autoantibody comes from “an aged mammal”, there is no requirement that the autoantibody actually comes from the patient being treated. In addition the patent discloses natural and monoclonal antinuclear autoantibody from an aged mammal, and a hybridoma cell line producing a monoclonal antinuclear autoantibody.

U.S. Patent Application 20050032128A1 discloses the use of anti-glycated CD59 antibodies for the treatment of diabetes.

### SUMMARY OF THE INVENTION

The instant inventors have previously been awarded U.S. Pat. No. 6,180,357, entitled “Individualized Patient Specific Anti-Cancer Antibodies” directed to a process for selecting individually customized anti-cancer antibodies which are useful in treating a cancerous disease. It is well recognized in the art that some amino acid sequence can be varied in a polypeptide without significant effect on the structure or function of the protein. In the molecular rearrangement of antibodies, modifications in the nucleic or amino acid sequence of the backbone region can generally be tolerated. These include, but are not limited to, substitutions (preferred are conservative substitutions), deletions or additions. Furthermore, it is within the purview of this invention to conjugate standard chemotherapeutic modalities, e.g. radionuclides, with the CDMAB of the instant invention, thereby focusing the use of said chemotherapeutics. The CDMAB can also be conjugated to toxins, cytotoxic moieties, enzymes e.g. biotin conjugated enzymes, or hematogenous cells, thereby forming an antibody conjugate.

This application utilizes the method for producing patient specific anti-cancer antibodies as taught in the '357 patent for isolating hybridoma cell lines which encode for cancerous disease modifying monoclonal antibodies. These antibodies can be made specifically for one tumor and thus make possible the customization of cancer therapy. Within the context of this application, anti-cancer antibodies having either cell-killing (cytotoxic) or cell-growth inhibiting (cytostatic) properties will hereafter be referred to as cytotoxic. These antibodies can be used in aid of staging and diagnosis of a cancer, and can be used to treat tumor metastases. These antibodies can also be used for the prevention of cancer by way of prophylactic treatment. Unlike antibodies generated according to traditional drug discovery paradigms, antibodies generated in this way may target molecules and pathways not previously shown to be integral to the growth and/or survival of malignant tissue. Furthermore, the binding affinity of these antibodies are suited to requirements for initiation of the cytotoxic events that may not be amenable to stronger affinity interactions.

The prospect of individualized anti-cancer treatment will bring about a change in the way a patient is managed. A likely clinical scenario is that a tumor sample is obtained at the time of presentation, and banked. From this sample, the tumor can be typed from a panel of pre-existing cancerous disease modifying antibodies. The patient will be conventionally staged but the available antibodies can be of use in further staging the patient. The patient can be treated immediately with the existing antibodies, and a panel of antibodies specific to the tumor can be produced either using the methods outlined herein or through the use of phage display libraries in conjunction with the screening methods herein disclosed. All the antibodies generated will be added to the library of anti-cancer antibodies since there is a possibility that other tumors can bear some of the same epitopes as the one that is being treated. The antibodies produced according to this method may be useful to treat cancerous disease in any number of patients who have cancers that bind to these antibodies.

In addition to anti-cancer antibodies, the patient can elect to receive the currently recommended therapies as part of a multi-modal regimen of treatment. The fact that the antibodies isolated via the present methodology are relatively non-toxic to non-cancerous cells allows for combinations of antibodies at high doses to be used, either alone, or in conjunction with conventional therapy. The high therapeutic index will also permit re-treatment on a short time scale that should decrease the likelihood of emergence of treatment resistant cells.

If the patient is refractory to the initial course of therapy or metastases develop, the process of generating specific antibodies to the tumor can be repeated for re-treatment. Furthermore, the anti-cancer antibodies can be conjugated to red blood cells obtained from that patient and re-infused for treatment of metastases. There have been few effective treatments for metastatic cancer and metastases usually portend a poor outcome resulting in death. However, metastatic cancers are usually well vascularized and the delivery of anti-cancer antibodies by red blood cells can have the effect of concentrating the antibodies at the site of the tumor. Even prior to metastases, most cancer cells are dependent on the host's blood supply for their survival and an anti-cancer antibody conjugated to red blood cells can be effective against in situ tumors as well. Alternatively, the antibodies may be conjugated to other hematogenous cells, e.g. lymphocytes, macrophages, monocytes, natural killer cells, etc.

There are five classes of antibodies and each is associated with a function that is conferred by its heavy chain. It is generally thought that cancer cell killing by naked antibodies are mediated either through antibody dependent cellular cytotoxicity or complement dependent cytotoxicity. For example murine IgM and IgG2a antibodies can activate human complement by binding the C1 component of the complement system thereby activating the classical pathway of complement activation which can lead to tumor lysis. For human antibodies the most effective complement activating antibodies are generally IgM and IgG1. Murine antibodies of the IgG2a and IgG3 isotype are effective at recruiting cytotoxic cells that have Fc receptors which will lead to cell killing by monocytes, macrophages, granulocytes and certain lymphocytes. Human antibodies of both the IgG1 and IgG3 isotype mediate ADCC.

Another possible mechanism of antibody mediated cancer killing may be through the use of antibodies that function to catalyze the hydrolysis of various chemical bonds in the cell membrane and its associated glycoproteins or glycolipids, so-called catalytic antibodies.

There are three additional mechanisms of antibody-mediated cancer cell killing. The first is the use of antibodies as a vaccine to induce the body to produce an immune response against the putative antigen that resides on the cancer cell. The second is the use of antibodies to target growth receptors and interfere with their function or to down regulate that receptor so that its function is effectively lost. The third is the effect of such antibodies on direct ligation of cell surface moieties that may lead to direct cell death, such as ligation of death receptors such as TRAIL R1 or TRAIL R2, or integrin molecules such as alpha V beta 3 and the like.

The clinical utility of a cancer drug is based on the benefit of the drug under an acceptable risk profile to the patient. In cancer therapy survival has generally been the most sought after benefit, however there are a number of other well-recognized benefits in addition to prolonging life. These other benefits, where treatment does not adversely affect survival, include symptom palliation, protection against adverse events, prolongation in time to recurrence or disease-free survival, and prolongation in time to progression. These criteria are generally accepted and regulatory bodies such as the U.S. Food and Drug Administration (F.D.A.) approve drugs that produce these benefits (Hirschfeld et al. Critical Reviews in Oncology/Hematolgy 42:137-143 2002). In addition to these criteria it is well recognized that there are other endpoints that may presage these types of benefits. In part, the accelerated approval process granted by the U.S. F.D.A. acknowledges that there are surrogates that will likely predict patient benefit. As of year-end (2003), there have been sixteen drugs approved under this process, and of these, four have gone on to full approval, i.e., follow-up studies have demonstrated direct patient benefit as predicted by surrogate endpoints. One important endpoint for determining drug effects in solid tumors is the assessment of tumor burden by measuring response to treatment (Therasse et al. Journal of the National Cancer Institute 92(3):205-216 2000). The clinical criteria (RECIST criteria) for such evaluation have been promulgated by Response Evaluation Criteria in Solid Tumors Working Group, a group of international experts in cancer. Drugs with a demonstrated effect on tumor burden, as shown by objective responses according to RECIST criteria, in comparison to the appropriate control group tend to, ultimately, produce direct patient benefit. In the pre-clinical setting tumor burden is generally more straightforward to assess and document. In that pre-clinical studies can be translated to the clinical setting, drugs that produce prolonged survival in pre-clinical models have the greatest anticipated clinical utility. Analogous to producing positive responses to clinical treatment, drugs that reduce tumor burden in the pre-clinical setting may also have significant direct impact on the disease. Although prolongation of survival is the most sought after clinical outcome from cancer drug treatment, there are other benefits that have clinical utility and it is clear that tumor burden reduction, which may correlate to a delay in disease progression, extended survival or both, can also lead to direct benefits and have clinical impact (Eckhardt et al. Developmental Therapeutics: Successes and Failures of Clinical Trial Designs of Targeted Compounds; ASCO Educational Book, 39^{th} Annual Meeting, 2003, pages 209-219). Using substantially the process of U.S. Pat. No. 6,180,357, and as disclosed in U.S. Pat. Nos. 6,794,494, Ser. No. 10/994,664, Ser. No. 11/067,366 and provisional Ser. No. 60/548,667, the contents of each of which are herein incorporated by reference, the mouse monoclonal antibodies, 10A304.7 and AR36A36.11.1 were obtained following immunization of mice with cells from human colon (10A304.7) or prostate (AR36A36.11.1) tumor tissue. The 10A304.7 and AR36A36.11.1 antigen was expressed on the cell surface of a wide range of human cell lines from different tissue origins. The breast cancer cell lines MDA-MB-231 (MB-231) and MCF-7, the colon cancer cell line SW1116, the prostate cancer cell line PC-3 and the ovarian cancer cell line OVCAR-3 were susceptible to the cytotoxic effect of 10A304.7 in vitro. The prostate cancer cell line LnCap was susceptible to the cytotoxic effects of AR36A36.11.1 in vitro.

The result of 10A304.7 cytotoxicity against breast cancer cells in vitro was further extended by demonstrating its anti-tumor activity in vivo (as disclosed in Ser. No. 10/994,664). 10A304.7 prevented tumor growth and reduced tumor burden in an in vivo model of human breast cancer. On day 56 post-implantation, 6 days after the last treatment dose, the mean tumor volume in the 10A304.7 treated group was 1 percent of the tumor volume in the isotype control treated group (p=0.0003, t-test). There were no clinical signs of toxicity throughout the study. Body weight measured at weekly intervals was a surrogate for health. There was no significant difference in body weight between the groups at the end of the treatment period (p=0.3512, t-test). Therefore 10A304.7 was well-tolerated and decreased the tumor burden in a human breast cancer xenograft model.

The result of AR36A36.11.1 cytotoxicity against prostate cancer cells in vitro was further extended by demonstrating its anti-tumor in vivo (as disclosed in Ser. No. 11/067,366). AR36A36.11.1 prevented tumor growth and reduced tumor burden in a preventative in vivo model of human prostate cancer. On day 41 post-implantation, 5 days after the last treatment dose, the mean tumor volume in the AR36A36.11.1 treated group was 14 percent of the tumor volume in the buffer control-treated group (p=0.0009, t-test). In a PC-3 prostate cancer xenograft model, body weight can be used as a surrogate indicator of disease progression (Wang et al. Int J Cancer, 2003). By the end of the study (day 41), control animals exhibited a 27% decrease in body weight from the onset of the study. By contrast, the group treated with AR36A36.11.1 had a significantly higher body weight than the control group (p=0.017). Overall, the AR36A36.11.1-treated group lost only 6% of its body weight, much less than the 27% lost by the buffer control group. Therefore AR36A36.11.1 was well-tolerated and decreased the tumor burden and cachexia in a human prostate cancer xenograft model.

In addition to its anti-prostate cancer effects, AR36A36.11.1 demonstrated anti-tumor activity against SW1116 colon cancer cells in a preventative in vivo tumor model (as disclosed in Ser. No. 11/067,366). On day 55 post-implantation, 5 days after the last treatment dose, the mean tumor volume in the AR36A36.11.1-treated group was 51 percent of the tumor volume in the buffer control-treated group (p=0.0055, t-test). There were no clinical signs of toxicity throughout the study. Body weight measured at weekly intervals was a surrogate for well-being and failure to thrive. There was no significant difference in body weight between the groups at the end of the treatment period (p=0.4409, t-test). Therefore AR36A36.11.1 was well-tolerated and decreased the tumor burden in a human colon cancer xenograft model.

In addition, AR36A36.11.1 demonstrated anti-tumor activity against MDA-MB-231 (MB-231) breast cancer in a preventative in vivo tumor model (as disclosed in Ser. No. 11/067,366). AR36A36.11.1 completely prevented tumor growth and reduced tumor burden. On day 56 post-implantation, 6 days after the last treatment dose, the mean tumor volume in the AR36A36.11.1 treated group was 0 percent of the tumor volume in the isotype control-treated group (p=0.0002, t-test). There were no clinical signs of toxicity throughout the study. Body weight measured at weekly intervals was a surrogate for well-being and failure to thrive. There was no significant difference in body weight between the groups at the end of the treatment period (p=0.0676, t-test). Therefore AR36A36.11.1 was well-tolerated and decreased the tumor burden in a human breast cancer xenograft model.

Also, AR36A36.11.1 demonstrated anti-tumor activity against MB-231 breast cancer in an established in vivo tumor model (as disclosed in Ser. No. 11/067,366). AR36A36.11.1 prevented tumor growth and reduced tumor burden in this established in vivo model of human breast cancer. On day 83 post-implantation, 2 days after the last treatment dose, the mean tumor volume in the AR36A36.11.1 treated group was 46% percent of the tumor volume in the buffer control-treated group (p=0.0038, t-test). This corresponds to a mean T/C of 32%. There were no clinical signs of toxicity throughout the study. Body weight measured at weekly intervals was a surrogate for well-being and failure to thrive. There was no significant difference in body weight between the groups at the end of the treatment period (p=0.6493, t-test).

In toto, this data demonstrates that the 10A304.7 and AR36A36.11.1 antigen is a cancer associated antigen and is expressed on human cancer cells, and is a pathologically relevant cancer target.

The present invention describes the development and use of 10A304.7 and AR36A36.11.1, developed by the process described in patent U.S. Pat. No. 6,180,357 and identified by, its effect, in a cytotoxic assay, in non-established and established tumor growth in animal models. This invention represents an advance in the field of cancer treatment in that it describes, for the first time, reagents that bind specifically to an epitope or epitopes present on the target molecule, CD59, and that also have in vitro cytotoxic properties against malignant tumor cells but not normal cells, and which also directly mediate inhibition of tumor growth in in vivo models of human cancer. This is an advance in relation to any other previously described anti-CD59 antibody, since none have been shown to have similar properties. A further advance is that inclusion of these antibodies in a library of anti-cancer antibodies will enhance the possibility of targeting tumors expressing different antigen markers by determination of the appropriate combination of different anti-cancer antibodies, to find the most effective in targeting and inhibiting growth and development of the tumors.

In all, this invention teaches the use of the 10A304.7 and AR36A36.11.1 antigen as a target for a therapeutic agent, that when administered can reduce the tumor burden of a cancer expressing the antigen in a mammal, and can also lead to a prolonged survival of the treated mammal. This invention also teaches the use of CDMAB (10A304.7 and AR36A36.11.1), and their derivatives, ligands thereof, e.g. cellular cytotoxicity inducing ligands thereof, and antigen binding fragments thereof, to target their antigen to prevent and reduce the tumor burden of a cancer expressing the antigen in a mammal, and to prolong the survival of a mammal bearing tumors that express this antigen. Furthermore, this invention also teaches the use of detecting the 10A304.7 and AR36A36.11.1 antigen in cancerous cells that can be useful for the diagnosis, prediction of therapy, and prognosis of mammals bearing tumors that express this antigen.

Accordingly, it is an objective of the invention to utilize a method for producing cancerous disease modifying antibodies (CDMAB) raised against cancerous cells derived from a particular individual, or one or more particular cancer cell lines, which CDMAB are cytotoxic with respect to cancer cells while simultaneously being relatively non-toxic to non-cancerous cells, in order to isolate hybridoma cell lines and the corresponding isolated monoclonal antibodies and antigen binding fragments thereof for which said hybridoma cell lines are encoded.

It is an additional objective of the invention to teach cancerous disease modifying antibodies, ligands and antigen binding fragments thereof.

It is a further objective of the instant invention to produce cancerous disease modifying antibodies whose cytotoxicity is mediated through antibody dependent cellular toxicity.

It is yet an additional objective of the instant invention to produce cancerous disease modifying antibodies whose cytotoxicity is mediated through complement dependent cellular toxicity.

It is still a further objective of the instant invention to produce cancerous disease modifying antibodies whose cytotoxicity is a function of their ability to catalyze hydrolysis of cellular chemical bonds.

A still further objective of the instant invention is to produce cancerous disease modifying antibodies which are useful for in a binding assay for diagnosis, prognosis, and monitoring of cancer.

Other objects and advantages of this invention will become apparent from the following description wherein are set forth, by way of illustration and example, certain embodiments of this invention.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 is a comparison of 10A304.7 versus positive and negative controls in a cytotoxicity assay.

FIG. 2. Western blots of MDA-MB-231 membrane proteins probed with AR36A36.11.1 (Panel A) and 110A304.7 (Panel B). Molecular weight markers are indicated on the left.

FIG. 3. Western blots probed with 10A304.7 (Panel A), AR36A36.11.1 (Panel B), IgG₂ₐ isotype control (8A304.7, Panel C), and IgG_{2b} isotype control (8B1B.1, Panel D). Lanes 1 through 4 are MDA-MB-231 membranes immunoprecipitated with 10A304.7 (Lane 1), AR36A36.11.1 (Lane 2), IgG₂ₐ isotype control (8A304.7, Lane 3), and IgG_{2b} isotype control (8B1B.1, Lane 4). Lane 5 is MDA-MB-231 membrane proteins, and Lane 6 is deglycosylated MDA-MB-231 membrane proteins, which were deglycosylated with PNGase F, sialidase A, o-glycanase, β(1-4) galactosidase and β-N-acetylglucosaminidase. Molecular weight markers are indicated on the left.

FIG. 4. Colloidal Blue stained (Panel A) and Western blot (Panel B) of MDA-MB-231 membranes immunoprecipitated with AR36A36.11.1 (Lane 1) and IgG₂ₐ isotype control (Lane 2). Molecular weight markers are indicated on the left.

FIG. 5. Western blot of MDA-MB-231 membrane proteins immunoprecipitated with mouse anti-human CD59 (MEM-43, Lane 1), AR36A36.11.1 (Lane 2) and IgG₂ₐ isotype control (8A3B.6, Lane 3) probed with 10A304.7 (Panel A), AR36A36.11.1 (Panel B), mouse anti-human CD59 (MEM-43, Panel C) and IgG₂ₐ isotype control (8A3B.6, Panel D). Molecular weight markers are indicated on the left.

FIGS. 6A-6C are a comparison of 10A304.7 and AR36A36.11.1 versus positive and negative controls on a human normal tissue microarray.

FIG. 7. Representative micrographs showing the binding pattern on normal human endometrium/secretary tissue obtained with 10A304.7 (A) or AR36A36.11.1 (B) or anti-actin (C) or the negative isotype control (D) from a normal human tissue microarray. 10A304.7 displayed negative staining while AR36A36.11.1 showed weak positive staining to the endothelium of blood vessels (see arrows). Magnification is 200×.

FIGS. 8A-8C are a comparison of 10A304.7 and AR36A36.11.1 versus positive and negative controls on a human various tumors tissue microarray.

FIG. 9. Representative micrographs showing the binding pattern on liver cholangiocarcinoma tissue obtained with 10A304.7 (A) or AR36A36.11.1 (B) or anti-actin (C) or the negative isotype control (D) from a human multi-tumor tissue microarray. 10A304.7 and AR36A36.11.1 showed positive staining to tumor cells. Magnification is 200×.

FIG. 10 is a summary of 10A304.7 binding on a human liver tumor and normal tissue microarray.

FIG. 11. Representative micrographs showing the binding pattern on hepatocellular carcinoma tissue obtained with 110A304.7 (A) or the isotype control antibody (B) and on non-neoplastic liver tissue obtained with 10A304.7 (C) or the isotype control antibody (D) from a human tissue microarray. 10A304.7 displayed strong positive staining for the tumor cells and negative staining on the normal tissue. Magnification is 200×.

### DETAILED DESCRIPTION OF THE INVENTION

In general, the following words or phrases have the indicated definition when used in the summary, description, examples, and claims.

The term “antibody” is used in the broadest sense and specifically covers, for example, single monoclonal antibodies (including agonist, antagonist, and neutralizing antibodies, de-immunized, murine, chimerized or humanized antibodies), antibody compositions with polyepitopic specificity, single chain antibodies, immunoconjugates and fragments of antibodies (see below).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma (murine or human) method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

“Antibody fragments” comprise a portion of an intact antibody, preferably comprising the antigen-binding or variable region thereof. Examples of antibody fragments include less than full length antibodies, Fab, Fab′, F(ab′)₂, and Fv fragments; diabodies; linear antibodies; single-chain antibody molecules; single-chain antibodies, single domain antibody molecules, fusion proteins, recombinant proteins and multispecific antibodies formed from antibody fragment(s).

An “intact” antibody is one which comprises an antigen-binding variable region as well as a light chain constant domain (CL) and heavy chain constant domains, C_{H}1, C_{H}2 and C_{H}3. The constant domains may be native sequence constant domains (e.g. human native sequence constant domains) or amino acid sequence variant thereof. Preferably, the intact antibody has one or more effector functions.

Depending on the amino acid sequence of the constant domain of their heavy chains, intact antibodies can be assigned to different “classes”. There are five-major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called a, d, e, ?, and μ, respectively. The subunit structures and three-dimensional configurations of different classes of immunoglobulins are well known.

Antibody “effector functions” refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; down regulation of cell surface receptors (e.g. B cell receptor; BCR), etc.

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express Fc?RIII only, whereas monocytes express Fc?RI, Fc?RII and Fc?RIII. FcR expression on hematopoietic cells in summarized is Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998).

“Effector cells” are leukocytes which express one or more FcRs and perform effector functions. Preferably, the cells express at least Fc?RIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source thereof, e.g. from blood or PBMCs as described herein.

The terms “Fc receptor” or “FcR” are used to describe a receptor that binds to the Fe region of an antibody. The preferred FcR is a native sequence human FcR. Moreover, a preferred FcR is one which binds an IgG antibody (a gamma receptor) and includes receptors of the Fc?RI, Fc?RII, and Fc? RIII subclasses, including allelic variants and alternatively spliced forms of these receptors. Fc?RII receptors include Fc?RIIA (an “activating receptor”) and Fc?RIIB (an “inhibiting receptor”), which have similar amino acid sequences that differ primarily in the cytoplasmic domains thereof. Activating receptor Fc?RIIA contains an immunoreceptor tyrosine-based activation motif (ITAM) in its cytoplasmic domain. Inhibiting receptor Fc?RIIB contains an immunoreceptor tyrosine-based inhibition motif (ITIM) in its cytoplasmic domain. (see review M. in Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., Eur. J. Immunol. 24:2429 (1994)).

“Complement dependent cytotoxicity” or “CDC” refers to the ability of a molecule to lyse a target in the presence of complement. The complement activation pathway is initiated by the binding of the first component of the complement system (C1q) to a molecule (e.g. an antibody) complexed with a cognate antigen. To assess complement activation, a CDC assay, e.g. as described in Gazzano-Santoro et al., J. Immunol. Methods 202:163 (1996), may be performed.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the >sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 2632 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework Region” or “FR” residues are those variable domain residues other than the hypervariable region residues as herein defined. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site. The Fab fragment also contains the constant domain of the light chain and the first constant domain (CH 1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments which have hinge cysteines between them. Other chemical couplings of antibody fragments are also known.

The “light chains” of antibodies from any vertebrate species can be assigned to one of two clearly distinct types, called kappa (?) and lambda (?), based on the amino acid sequences of their constant domains.

“Single-chain Fv” or “scFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains which enables the scFv to form the desired structure for antigen binding. For a review of scFv see Plückthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a variable heavy domain (V_{H}) connected to a variable light domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other protcinaceous or nonproteinaceous solutes. Isolated antibody includes the antibody in situ within recombinant cells since at least one component of the antibody's natural environment will not be present. Ordinarily, however, isolated antibody will be prepared by at least one purification step.

An antibody “which binds” an antigen of interest, e.g. CD59 antigen, is one capable of binding that antigen with sufficient affinity such that the antibody is useful as a therapeutic agent in targeting a cell expressing the antigen. Where the antibody is one which binds CD59, it will usually preferentially bind CD59 as opposed to other receptors, and does not include incidental binding such as non-specific Fc contact, or binding to post-translational modifications common to other antigens and may be one which does not significantly cross-react with other proteins. Methods, for the detection of an antibody that binds an antigen of interest, are well known in the art and can include but are not limited to assays such as FACS, cell ELISA and Western blot.

As used herein, the expressions “cell”, “cell line”, and “cell culture” are used interchangeably, and all such designations include progeny. It is also understood that all progeny may not be precisely identical in DNA content, due to deliberate or inadvertent mutations. Mutant progeny that have the same function or biological activity as screened for in the originally transformed cell are included. It will be clear from the context where distinct designations are intended.

“Treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented. Hence, the mammal to be treated herein may have been diagnosed as having the disorder or may be predisposed or susceptible to the disorder.

The terms “cancer” and “cancerous” refer to or describe the physiological condition in mammals that is typically characterized by unregulated cell growth or death. Examples of cancer include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

A “chemotherapeutic agent” is a chemical compound useful in the treatment of cancer. Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carnomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2?-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL®, Bristol-Myers Squibb Oncology, Princeton, N.J.) and docetaxel (TAXOTERE®, Aventis, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

“Mammal” for purposes of treatment refers to any animal classified as a mammal, including humans, mice, SCID or nude mice or strains of mice, domestic and farm animals, and zoo, sports, or pet animals, such as sheep, dogs, horses, cats, cows, etc. Preferably, the mammal herein is human.

“Oligonucleotides” are short-length, single- or double-stranded polydeoxynucleotides that are chemically synthesized by known methods (such as phosphotriester, phosphite, or phosphoramidite chemistry, using solid phase techniques such as described in EP 266,032, published 4 May 1988, or via deoxynucleoside H-phosphonate intermediates as described by Froehler et al., Nucl. Acids Res., 14:5399-5407, 1986. They are then purified on polyacrylamide gels.

Unless indicated otherwise, the term “CD59” when used herein refers to the mammalian glycosyl phosphatidylinositol (GPI)-anchored membrane glycoprotein also referred to as MEM-43 antigen, membrane inhibitor of reactive lysis (MIRL), H19, membrane attack complex-inhibitory factor (MACIF), homologous restriction factor with m.w. 20,000 (HRF20) and protectin (Walsh, Tone et al. 1992).

“Chimeric” antibodies are immunoglobulins in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567 and Morrison et al, Proc. Natl. Acad. Sci. USA, 81:6851-6855 (1984)).

“Humanized” forms of non-human (e.g. murine) antibodies are specific chimeric immunoglobulins, immunoglobulin chains or fragments thereof (such as Fv, Fab, Fab′, F(ab)₂ or other antigen-binding subsequences of antibodies) which contain minimal sequence derived from non-human immunoglobulin. For the most part, humanized antibodies are human immunoglobulins (recipient antibody) in which residues from the complementarity determining regions (CDRs) of the recipient antibody are replaced by residues from the CDRs of a non-human species (donor antibody) such as mouse, rat or rabbit having the desired specificity, affinity and capacity. In some instances, Fv framework region (FR) residues of the human immunoglobulin are replaced by corresponding non-human FR residues. Furthermore, the humanized antibody may comprise residues which are found neither in the recipient antibody nor in the imported CDR or FR sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR residues are those of a human immunoglobulin consensus sequence. The humanized antibody optimally also will comprise at least a portion of an immunoglobulin constant region (Fc), typically that of a human immunoglobulin.

“De-immunized” antibodies are immunoglobulins that are non-immunogenic, or less immunogenic, to a given species. De-immunization can be achieved through structural alterations to the antibody. Any de-immunization technique known to those skilled in the art can be employed. One suitable technique for de-immunizing antibodies is described, for example, in WO 00/34317 published Jun. 15, 2000.

“Homology” is defined as the percentage of residues in the amino acid sequence variant that are identical after aligning the sequences and introducing gaps, if necessary, to achieve the maximum percent homology. Methods and computer programs for the alignment are well known in the art.

Throughout the instant specification, hybridoma cell lines, as well as the isolated monoclonal antibodies which are produced therefrom, are alternatively referred to by their internal designation, 10A304.7 or AR36A36.11.1 or Depository Designation, ATCC PTA-5065 or IDAC 280104-02 respectively.

As used herein “ligand” includes a moiety which exhibits binding specificity for a target antigen, and which may be an intact antibody molecule and any molecule having at least an antigen-binding region or portion thereof (i.e., the variable portion of an antibody molecule), e.g., an Fv molecule, Fab molecule, Fab′ molecule, F(ab′).sub.2 molecule, a bispecific antibody, a fusion protein, or any genetically engineered molecule which specifically recognizes and binds the antigen bound by the isolated monoclonal antibody produced by the hybridoma cell line designated as, ATCC PTA-5065 or IDAC 280104-02 (the ATCC PTA-5065 or IDAC 280104-02 antigen).

As used herein “antigen-binding region” means a portion of the molecule which recognizes the target antigen.

As used herein “competitively inhibits” means being able to recognize and bind a determinant site to which the monoclonal antibody produced by the hybridoma cell line designated as ATCC PTA-5065 or IDAC 280104-02, (the ATCC PTA-5065 or IDAC 280104-02 antibody) is directed using conventional reciprocal antibody competition assays. (Belanger L., Sylvestre C. and Dufour D. (1973), Enzyme linked immunoassay for alpha fetoprotein by competitive and sandwich procedures. Clinica Chimica Acta 48, 15).

As used herein “target antigen” is the ATCC PTA-5065 or IDAC 280104-02 antigen or portions thereof.

As used herein, an “immunoconjugate” means any molecule or ligand such as an antibody chemically or biologically linked to a cytotoxin, a radioactive agent, enzyme, toxin, an anti-tumor drug or a therapeutic agent. The antibody may be linked to the cytotoxin, radioactive agent, anti-tumor drug or therapeutic agent at any location along the molecule so long as it is able to bind its target. Examples of immunoconjugates include antibody toxin chemical conjugates and antibody-toxin fusion proteins.

As used herein, a “fusion protein” means any chimeric protein wherein an antigen binding region is connected to a biologically active molecule, e.g., toxin, enzyme, or protein drug.

In order that the invention herein described may be more fully understood, the following description is set forth.

The present invention provides ligands (i.e., ATCC PTA-5065 or IDAC 280104-02 ligands) which specifically recognize and bind the ATCC PTA-5065 or IDAC 280104-02 antigen.

The ligand of the invention may be in any form as long as it has an antigen-binding region which competitively inhibits the immunospecific binding of the monoclonal antibody produced by hybridoma ATCC PTA-5065 or IDAC 280104-02 to its target antigen. Thus, any recombinant proteins (e.g., fusion proteins wherein the antibody is combined with a second protein such as a lymphokine or a tumor inhibitory growth factor) having the same binding specificity as the ATCC PTA-5065 or IDAC 280104-02 antibody fall within the scope of this invention.

In one embodiment of the invention, the ligand is the ATCC PTA-5065 or IDAC 280104-02 antibody.

In other embodiments, the ligand is an antigen binding fragment which may be a Fv molecule (such as a single chain Fv molecule), a Fab molecule, a Fab′ molecule, a F(ab′)2 molecule, a fusion protein, a bispecific antibody, a heteroantibody or any recombinant molecule having the antigen-binding region of the ATCC PTA-5065 or IDAC 280104-02 antibody. The ligand of the invention is directed to the epitope to which the ATCC PTA-5065 or IDAC 280104-02 monoclonal antibody is directed.

The ligand of the invention may be modified, i.e., by amino acid modifications within the molecule, so as to produce derivative molecules. Chemical modification may also be possible.

Derivative molecules would retain the functional property of the polypeptide, namely, the molecule having such substitutions will still permit the binding of the polypeptide to the ATCC PTA-5065 or IDAC 280104-02 antigen or portions thereof.

These amino acid substitutions include, but are not necessarily limited to, amino acid substitutions known in the art as “conservative”.

For example, it is a well-established principle of protein chemistry that certain amino acid substitutions, entitled “conservative amino acid substitutions,” can frequently be made in a protein without altering either the conformation or the function of the protein.

Such changes include substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine (S) for threonine (T) and vice versa. Other substitutions can also be considered conservative, depending on the environment of the particular amino acid and its role in the three-dimensional structure of the protein. For example, glycine (G) and alanine (A) can frequently be interchangeable, as can alanine and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered “conservative” in particular environments.

Given an antibody, an individual ordinarily skilled in the art can generate a competitively inhibiting ligand, for example a competing antibody, which is one that recognizes the same epitope (Belanger et al., 1973). One method could entail immunizing with an immunogen that expresses the antigen recognized by the antibody. The sample may include but is not limited to tissue, isolated protein(s) or cell line(s). Resulting hybridomas could be screened using a competing assay, which is one that identifies antibodies that inhibit the binding of the test antibody, such as ELISA, FACS or immunoprecipiation. Another method could make use of phage display libraries and panning for antibodies that recognize said antigen (Rubinstein et al., 2003). In either case, hybridomas would be selected based on their ability to out-compete the binding of the original antibody to its target antigen. Such hybridomas would therefore possess the characteristic of recognizing the same antigen as the original antibody and more specifically would recognize the same epitope.

### EXAMPLE 1

In Vitro Cytotoxicity

10A304.7 monoclonal antibody was produced by culturing the hybridoma in CL-1000 flasks (BD Biosciences, Oakville, ON) with collections and reseeding occurring twice/week. Standard antibody purification procedures with Protein G Sepharose 4 Fast Flow (Amersham Biosciences, Baie d∝Urfé, QC) were followed. It is within the scope of this invention to utilize monoclonal antibodies that are humanized, chimerized or murine.

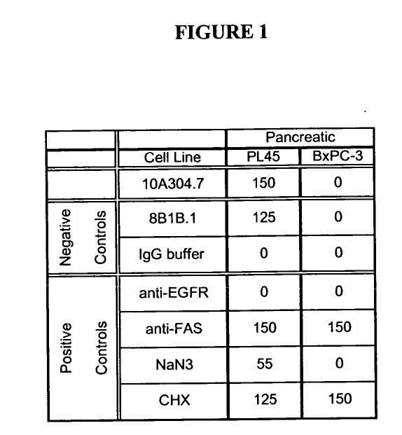

10A304.7 was compared to a number of both positive (anti-EGFR antibody (C225, IgG1, kappa, 5 micrograms/mL, Cedarlane, Hornby, ON; anti-FAS, IgM, kappa, 10 micrograms/mL, eBiosciences, San Diego, Calif.), cycloheximide (CHX, 0.5 micromolar, Sigma, Oakville, ON), and NaN₃ (0.1%, Sigma, Oakville, ON)) controls, and a negative isotype control 8B1B.1 (anti-bluetongue virus, purified in-house), as well as a buffer diluent control in a cytotoxicity assay (FIG. 1). 10A304.7 and isotype control antibody were assessed at 10 micrograms/mL on two pancreatic cancer cell lines (BxPC-3, PL45). Both cell lines were obtained from the ATCC (Manassas, Va.). Calcein AM was obtained from Molecular Probes (Eugene, Oreg.). The assays were performed according to the manufacturer's instructions with the changes outlined below. Cells were plated before the assay at the predetermined appropriate density. After 2 days, 100 microliters of purified antibody or controls were diluted into media, and then transferred to the cell plates and incubated in a 5 percent CO₂ incubator for 5 days. The plates were then emptied by inverting and blotted dry. Room temperature DPBS containing MgCl₂ and CaCl₂ was dispensed into each well from a multichannel squeeze bottle, tapped 3 times, emptied by inversion and then blotted dry. Fifty microlitres of the fluorescent calcein dye diluted in DPBS containing MgCl₂ and CaCl₂ was added to each well and incubated at 37° C. in a 5 percent CO₂, incubator for 30 minutes. The plates were read in a Perkin-Elmer HTS7000 fluorescence plate reader and the data was analyzed in Microsoft Excel and the results were tabulated in FIG. 1. Each antibody received a score between 5 and 50 based on the average cytotoxicity observed in four experiments tested in triplicate, and a score between 25 and 100 based on the variability observed between assays. The sum of these two scores (the cytotoxicity score) is presented in FIG. 1. A cytotoxicity score of greater than or equal to 55 was considered to be positive on the cell line tested. 10A304.7 had no cytotoxic effect on the BxPC-3 cell line, as previously disclosed in U.S. Pat. No. 6,794,494. The 10A304.7 antibody was found to have specific cytotoxicity, above both the buffer and isotype negative controls, in the pancreatic PL45 cell line. The buffer demonstrated no measureable cytotoxicity. In this particular experiment, the 8B1B.1 isotype control showed higher than normal cytotoxicity against the PL45 cell line, which may be a function of the variability inherent in biological assays. Although the effect of the isotype control was high, results obtained with 10A304.7 were consistently higher in each experiment. These results demonstrate that 10A304.7 has functional specificity, and can target cancer cells derived from human pancreatic cancer.

### EXAMPLE 2

Identification of Binding Proteins by Western Blotting

To identify the antigens recognized by the antibodies 10A304.7 and AR36A36.11.1, cell membranes expressing the antigens were subjected to gel electrophoresis and transferred to membranes using Western blotting to determine the proteins bound by these antibodies.

1. Membrane Preparation

Previous work demonstrated that both 10A304.7 and AR36A36.11.1 showed efficacy against breast cancer as exemplified by the cell line MDA-MB-231 (MB-231) grown as xenografts in severe combined immunodeficient (SCID) mice. Accordingly, MB-231 membrane preparations were used for antigen identification. Total cell membranes were prepared from confluent cultures of MB-231 cells. Media was removed from cell stacks and the cells were washed in phosphate buffered saline (PBS). Cells were dissociated with dissociation buffer (Gibco-BRL, Grand Island, N.Y.) for 20 minutes at 37° C. on a platform shaker. Cells were collected and centrifuged at 900 g for 10 minutes at 4° C. After centrifugation, cell pellets were resuspended in PBS and centrifuged again at 900 g for 10 minutes at 4° C. to wash. Supernatant was poured off and pellets were stored at −80° C. Cell pellets were resuspended in homogenization buffer containing 1 tablet per 50 mL of Complete protease inhibitor cocktail (Roche, Laval QC) at a ratio of 3 mL buffer per gram of cells. The cell suspension was subjected to homogenization using a polytron homogenizer on ice in order to lyse the cells. The cell homogenate was centrifuged at 15,000 g for 10 minutes at 4° C. to remove the nuclear particulate. Supernatant was harvested, divided into tubes and then centrifuged at 75,600 g for 90 minutes at 4° C. Supernatant was carefully removed from the tubes and each membrane pellet was resuspended in approximately 5 mL homogenization buffer. The resuspended pellets from all tubes were combined together in one tube and centrifuged at 75,600 g for 90 minutes at 4° C. Supernatant from the tubes was carefully removed, and the pellets were weighed. Solubilization buffer containing 1 percent Triton X-100 was added to the pellets at a ratio of 3 mL buffer per gram of membrane pellet. Membranes were solubilized by shaking on a platform shaker at 300 rpm for 1 hour on ice. The membrane solution was centrifuged at 75,600 g to pellet insoluble material. The supernatant containing the solubilized membrane proteins was carefully removed from tubes, assayed for protein content, and stored at −80° C.

2. Western Blots

Membrane proteins were separated by SDS-polyacrylamide gel electrophoresis. 20 micrograms of MB-231 membrane protein was mixed with non-reducing SDS-PAGE sample buffer and loaded onto a lane of duplicate 4-20 percent gradient SDS-PAGE gels (Bio-Rad, Mississauga, ON). A sample of unstained molecular weight markers (Invitrogen, Burlington, ON) was run in a reference lane. Electrophoresis was carried out at 100 V for 10 minutes, followed by 150 V until the dye front from the sample buffer had run off the gels. Proteins were transferred from the gels to PVDF membranes (Millipore, Billerica, Mass.) by electroblotting for 16 hour at 40 V. Following transfer, membranes were blocked with 5 percent skim milk powder in Tris-buffered saline containing 0.5 percent Tween-20 (TBST) for 1 hour. Membranes were washed three times with TBST and then incubated with either 5 micrograms/mL 10A304.7 or 5 micrograms/mL AR36A36.11.1 diluted in 5 percent skim milk powder in TBST for 2 hours. After washing 3 times with TBST, membranes were incubated with goat anti-mouse IgG (Fc) conjugated to horseradish peroxidase (HRP) from Jackson Immunologicals (West Grove Pa.). This incubation was followed by washing 3 times with TBST, followed by incubation with ECL Plus western detection reagents (Amersham Biosciences, Baie d'Urfé, QC). Blots were exposed to chemiluminescent film (Kodak, Cedex, France) and developed using an X-ray medical processor.

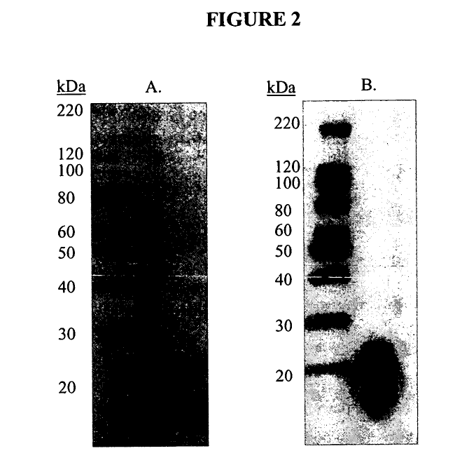

FIG. 2 shows AR36A36.11.1 (Panel A) and 10A304.7 (Panel B) strongly bind to protein(s) in the lower region of the membrane. By comparison to the molecular weight standards, the antibodies bind to protein(s) approximately 20 kDa. Both antibodies bind to MB-231 membranes in a similar pattern.

### EXAMPLE 3

Cross-Immunoprecipitation and Deglycosylation of Antigens Bound by AR36A36.11.1 and 10A304.7

To determine if the antigens bound by 10A304.7 and AR36A36.11.1 were identical, MB-231 membranes were cross-immunoprecipitated with the two antibodies. The appropriate isotype controls (8A3B.6 for IgG₂ₐ, the isotype of AR36A36.11.1, and 8B1B.1 for IgG_{2b}, the isotype of 10A304.7) were included to ensure that any reactivity was specific to the functional antibodies.

1. Immunoprecipitation

200 micrograms of each antibody was diluted to 1 mL in 0.1 M sodium phosphate, pH 6.0. 100 microliters of protein G sepharose beads (Amersham Biosciences, Baie d'Urfé, QC) per antibody was washed 3 times in 1 mL 0.1 M sodium phosphate, pH 6.0. The diluted antibodies were added to the aliquots of beads and incubated for 1 hour at room temperature with rotational mixing. The unbound antibody was removed by spinning in a microcentrifuge at 14,000 rpm for 20 seconds, and then aspirating off the supernatant. The antibody coated beads were washed 3 times with 1 mL 0.1 M sodium phosphate, pH 7.4, followed by 2 washes with 1 mL 0.2 M triethanolamine, pH 8.2. The antibody bound beads were resuspended in 1 mL 0.2 M triethanolamine, pH 8.2, and then chemically cross-linked by adding 5.2 mg dimethylpimelimidate (Sigma, Oakville, ON) and incubating with rotational mixing for 1 hour. The antibody cross-linked beads were rinsed once with 1.5 mL 50 mM Tris, pH 7.5, followed by incubation with 1 mL 50 mM Tris, pH 7.5 for 30 minutes at room temperature with rotational mixing. The beads were washed 3 times with PBS, then resuspended in 100 microliters phosphate buffered saline containing 0.02 percent sodium azide and stored at 4° C.

Each of the four antibodies was used for immunoprecipitation with MB-231 membranes, AR36A36.11.1, 10A304.7, 8A3B.6 and 8B1B.1, using the conjugated beads described above. 200 micrograms of MB-231 membrane preparation was diluted to 1 mL with normal lysis buffer (50 mM Tris, pH 7.4, 150 mM sodium chloride, 2 mM EDTA, 1 percent Triton X-100, 50 mM sodium fluoride, 2 mM sodium orthovanadate and 1× protease inhibitor cocktail) per antibody. 50 micrograms of antibody-conjugated beads per antibody was added to the diluted MB-231 membranes and incubated at 4° C. for 2 hours rotating end-over-end. The immunocomplex bound beads were washed 3 times with normal lysis buffer and once with PBS. The immunocomplex bound beads were resuspended in phosphate buffered saline and stored at 4° C. until ready for use.

2. Deglycosylation

To test the role of carbohydrate groups on antigen binding of AR36A36.11.1 and 10A304.7, MB-231 membranes were deglycosylated. 100 micrograms of MB-231 membrane was incubated with PNGase F, sialidase A, o-glycanase, β(1-4) galactosidase and β-N-acetylglucosaminidase from GLYKO Enzymatic Digestion kit (ProZyme, San Leandro, Calif.) as per manufacturer's instructions under non-reducing conditions. An additional 100 micrograms aliquot of MB-231 membrane was incubated with only the deglycosylation buffers to act as a glycosylated control reaction.

3. Western Blots

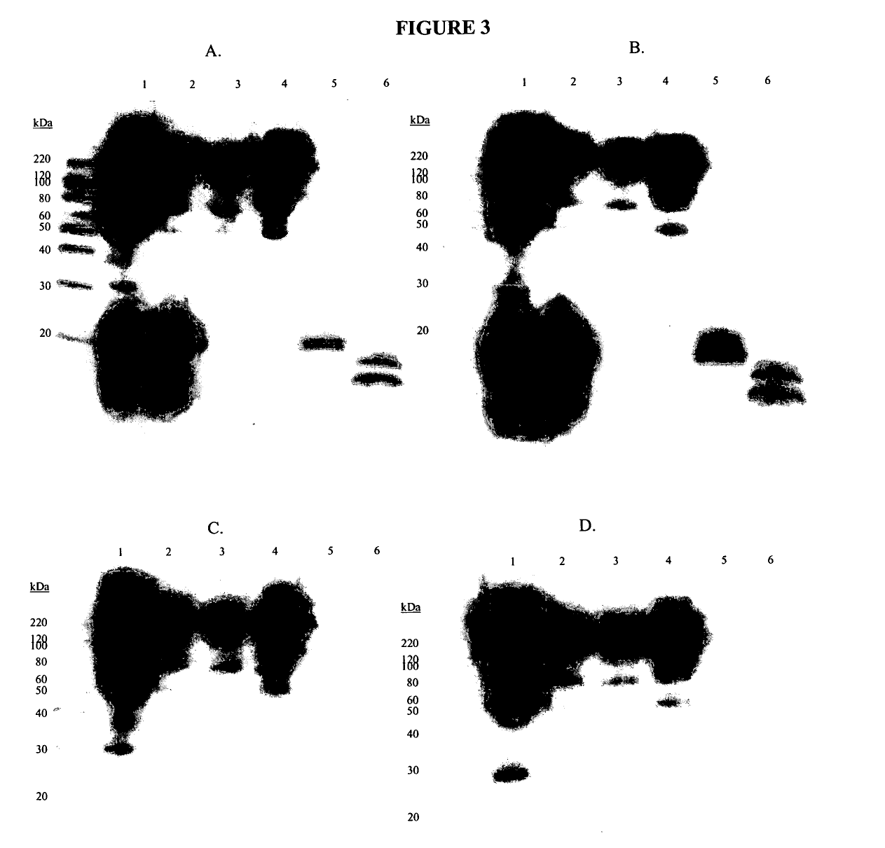

The 10A304.7, AR36A36.11.1, IgG₂ₐ isotype and IgG_{2b} isotype immunoprecipitated MB-231 membranes, as well as the glycosylated and deglycosylated MB-231 membranes, were combined with non-reducing SDS-PAGE sample buffer and loaded onto quadruplicate 12 percent SDS-PAGE gels (Bio-Rad, Mississauga, ON). Unstained molecular weight markers were loaded in reference lanes. Membranes were separated by SDS-PAGE followed by Western blotting as described in Example 2. FIG. 3 demonstrates the binding of 10A304.7 (Panel A), AR36A36.11.1 (Panel B), IgG₂ₐ isotype control (Panel C) and IgG_{2b} isotype control (Panel D) to MB-231 membranes immunoprecipitated with 10304.7 (Lane 1), MB-231 membranes immunoprecipitated with AR36A36.11.1 (Lane 2), MB-231 membranes immunoprecipitated with IgG₂ₐ isotype control (Lane 3), MB-231 membranes immunoprecipitated with IgG_{2b} isotype control (Lane 4), MB-231 glycosylated membranes (Lane 5) and MB-231 deglycosylated membranes (Lane 6). Panels A and B have identical binding in all lanes, indicating that AR36A36.11.1 and 10A304.7 recognize the same antigen. The large smear in the 20 kDa region of MB-231 membranes immunoprecipitated with 10A304.7 and AR36A36.11.1 (Lanes 1 and 2, respectively) appear only when probed with 10A304.7 and AR36A36.11.1 (Panels A and B, respectively) and not when probed with isotype controls (Panels C and D), indicating the binding in that region is specific to the functional antibodies. There is no reactivity in the 20 kDa region when MB-231 membranes are immunoprecipitated with the isotype controls (Lanes 3 and 4), further indicating the specificity of the functional antibodies for the antigen. Both 10A304.7 and AR36A36.11.1 bind to a doublet in the 20 kDa region of the glycosylated MB-231 membranes (Lane 5). There is a shift in reactivity when the membranes are deglycosylated (Lane 6), indicating the antigen is glycosylated but that the carbohydrate groups are not essential for antigen binding.

### EXAMPLE 4

Identification of Antigens Bound by 10A304.7 and AR36A36.11.1

1. Immunoprecipitation

The antigen bound by AR36A36.11.1 was isolated from MB-231 cells by immunoprecipitation. 1 mL of Protein G Sepharose (Amersham Bioscience, Baie d'Urfé, QC) was cross-linked to 2 mg of antibody following the same protocol disclosed in Example 3, scaling up accordingly. Both AR36A36.11.1 and 8A3B.6 were cross-linked.

A 10 mg aliquot of MB-231 was diluted to 10 mL with RIPA buffer (50 mM Tris-HCl, pH 7.4, 150 mM NaCl, 1 percent NP-40, 0.5 percent sodium deoxycholate, 0.1 percent SDS, 2 mM sodium orthovanadate and 1× protease inhibitor cocktail). 3 mL of Sepharose 4B (Sigma, Oakville, ON) was added and incubated at 4° C. for 2 hours rotating end-over-end. 60 microliters of both antibody-conjugated beads were concurrently incubated with 0.5 mg/mL of BSA diluted in 0.1 M NaH₂PO₄, pH 7.4, at 4° C. for 2 hours rotating end-over-end. The antibody-conjugated beads were washed twice with RIPA buffer, and then drained. For the immunoprecipitation, the pre-cleared MB-231 membranes were removed from the Sepharose 4B beads and added to the 8A3B.6-conjugated beads, and incubated for 2 hours at 4° C. rotating end-over-end. Following incubation with the isotype control, the diluted membrane prep was incubated with the AR36A36.11.1-conjugated beads for 2 hours at 4° C. rotating end-over-end. Both aliquots of beads were then washed twice with RIPA buffer and once with PBS

2. SDS-PAGE

The immunoprecipitated beads were resuspended in 30 microliters SDS-PAGE sample buffer and boiled for 3 minutes, followed by cooling to room temperature. 21 microliters of the samples was loaded into one lane of a 12 percent SDS-PAGE gel (Bio-Rad, Mississauga, ON), and the remaining 7 microliters into another lane. Protein standards and prestained molecular weight markers (Invitrogen, Burlington, ON) were also included on the gel. The gel was run at 100 V for 10 minutes, then 150 V until the leading dye front had run off the gel. The gel was cut along the lane loaded with prestained molecular weight marker. The part of the gel loaded with 21 microliters was stained with Colloidal Blue, and the other part of the gel was transferred to PVDF membrane for Western blotting with AR36A36.11.1, following the protocol disclosed in Example 2.

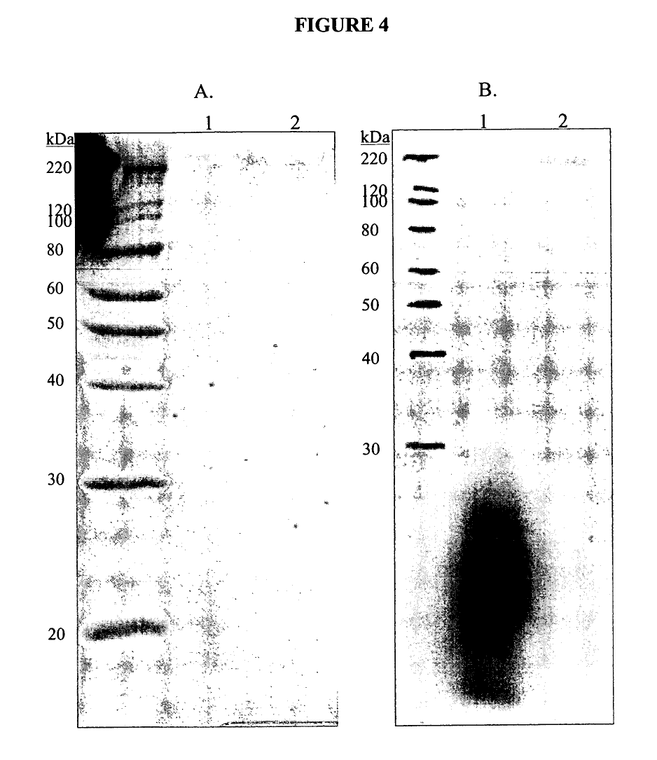

The Colloidal Blue staining reagents (Invitrogen, Burlington, ON) were prepared as per manufacturer's instructions, and the gel was incubated in the stain overnight shaking at room temperature. The gel was incubated in water to remove background staining for 2 hours. FIG. 4 demonstrates MB-231 membranes immunoprecipitated with AR36A36.11.1 (Lane 1) and 8A3B.6 IgG₂ₐ isotype control (Lane 2). The faint doublet at 20 kDa on the stained gel (Panel A, Lane 1) corresponds to the reactivity observed in the Western blot (Panel B, Lane 1). These two bands were extracted from the stained gel using sterile glass Pasteur pipettes, along with the corresponding areas on the gel from Lane 2 and an area in which no protein had been loaded for background controls.

3. Mass Spectrometry

The extracted gel pieces were digested using an In-Gel Tryptic Digestion kit (Pierce, Rockford, Ill.). A portion of each sample was spotted onto an H4 ProteinChip Array (Ciphergen, Freemont, Calif.) using CHCA matrix. The array chips were analyzed on a Ciphergen SELDI/MS using ProteinChip Software (Ciphergen). The unique peptide peaks from the bottom band of the doublet from the AR36A36.11.1 immunoprecipitation were 1540.6, 1649.6, 1741.6, 1778.1 and 2015.2 Da. Using the ProFound peptide mapping database (Rockefeller University), CD59 was identified as the source of these peptides with a probability of 1.00 and an estimated Z value of 1.92. CD59 contains the peptides 1539.6, 1648.6 and 2014.1 Da.

4. Confirmation

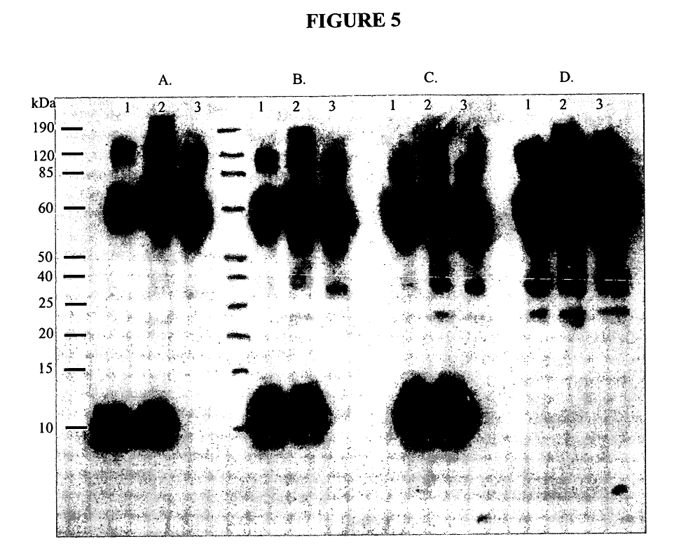

To confirm that the antigen recognized by AR36A36.11.1 and 10A304.7 was CD59, MB-231 membranes were cross-immunoprecipitated with a commercial research anti-CD59 antibody. 50 micrograms of mouse anti-human CD59, clone MEM-43 (IgG₂ₐ) (Serotec, Raleigh, N.C.) was cross-linked to 25 microliters Protein G Sepharose beads, as described in Example 3. Three 150 micrograms aliquots of MB-231 membranes were immunoprecipitated with 25 microliters of beads conjugated to anti-CD59, AR36A36.11.1 or 8A3B.6 IgG₂ₐ isotype control, as described in Example 3. The beads were resuspended in 45 microliters PBS, then 15 microliters of SDS-PAGE sample buffer was added and the samples were boiled for 3 minutes. After cooling to room temperature, the samples were loaded 15 microliters per well onto a 4-20 percent SDS-PAGE gel (Bio-Rad, Mississauga, ON). Electrophoresis and Western blotting were carried out as described above. The membranes were incubated with 3.33 micrograms/mL anti-CD59 (MEM-43), 5 micrograms/mL of AR36A36.11.1, 5 micrograms/mL of 10A304.7 or 5 micrograms/mL 8A3B.6 IgG₂ₐ isotype control diluted in 5 percent skim milk for 2 hours. FIG. 5 shows the Western blots of MDA-MB-231 membrane proteins immunoprecipitated with mouse anti-human CD59 (MEM-43, Lane 1), AR36A36.11.1 (Lane 2) and IgG₂ₐ isotype control (8A3B.6, Lane 3) probed with 10A304.7 (Panel A), AR36A36.11.1 (Panel B), mouse anti-human CD59 (MEM-43, Panel C) and IgG₂ₐ isotype control (8A3B.6, Panel D). The reactivity in the higher molecular weight regions appear when the membrane is probed with isotype control (Panel D) and are therefore regarded as background. The blots incubated with 10A304.7 (Panel A), AR36A36.11.1 (Panel B) and anti-CD59 (Panel C) have identical staining in all three lanes; a small molecular weight band specifically reacts with membranes immunoprecipitated with AR36A36.11.1 and anti-CD59. This confirms that the antigen recognized by 10A304.7 and AR36A36.11.1 is CD59.

### EXAMPLE 5

Normal Human Tissue Staining

IHC studies were conducted to characterize the 10A304.7 and AR36A36.11.1 antigen distribution in humans. IHC optimization studies were performed previously in order to determine the conditions for further experiments.

Tissue sections were deparaffinized by drying in an oven at 58° C. for 1 hour and dewaxed by immersing in xylene 5 times for 4 minutes each in Coplin jars. Following treatment through a series of graded ethanol washes (100%-75%) the sections were re-hydrated in water. The slides were immersed in 10 mM citrate buffer at pH 6 (Dako, Toronto, Ontario) then microwaved at high, medium, and low power settings for 5 minutes each and finally immersed in cold PBS. Slides were then immersed in 3% hydrogen peroxide solution for 6 minutes, washed with PBS three times for 5 minutes each, dried and then incubated with Universal blocking solution (Dako, Toronto, Ontario) for 5 minutes at room temperature. 10A304.7, AR36A36.11.1, monoclonal mouse anti-vimentin (Dako, Toronto, Ontario) or isotype control antibody (directed towards Aspergillus niger glucose oxidase, an enzyme which is neither present nor inducible in mammalian tissues; Dako, Toronto, Ontario) were diluted in antibody dilution buffer (Dako, Toronto, Ontario) to its working concentration (5 micrograms/mL for each antibody) and incubated for 1 hour at room temperature. The slides were washed with PBS 3 times for 5 minutes each. Immunoreactivity of the primary antibodies was detected/visualized with HRP conjugated secondary antibodies as supplied (Dako Envision System, Toronto, Ontario) for 30 minutes at room temperature. Following this step the slides were washed with PBS 3 times for 5 minutes each and a color reaction developed by adding DAB (3,3′-diaminobenzidine tetrahydrachloride, Dako, Toronto, Ontario) chromogen substrate solution for immunoperoxidase staining for 10 minutes at room temperature. Washing the slides in tap water terminated the chromogenic reaction. Following counterstaining with Meyer's Hematoxylin (Sigma Diagnostics, Oakville, ON), the slides were dehydrated with graded ethanols (75-100%) and cleared with xylene. Using mounting media (Dako Faramount, Toronto, Ontario) the slides were coverslipped. Slides were microscopically examined using an Axiovert 200 (Ziess Canada, Toronto, ON) and digital images acquired and stored using Northern Eclipse Imaging Software (Mississauga, ON). Results were read, scored and interpreted by a histopathologist.

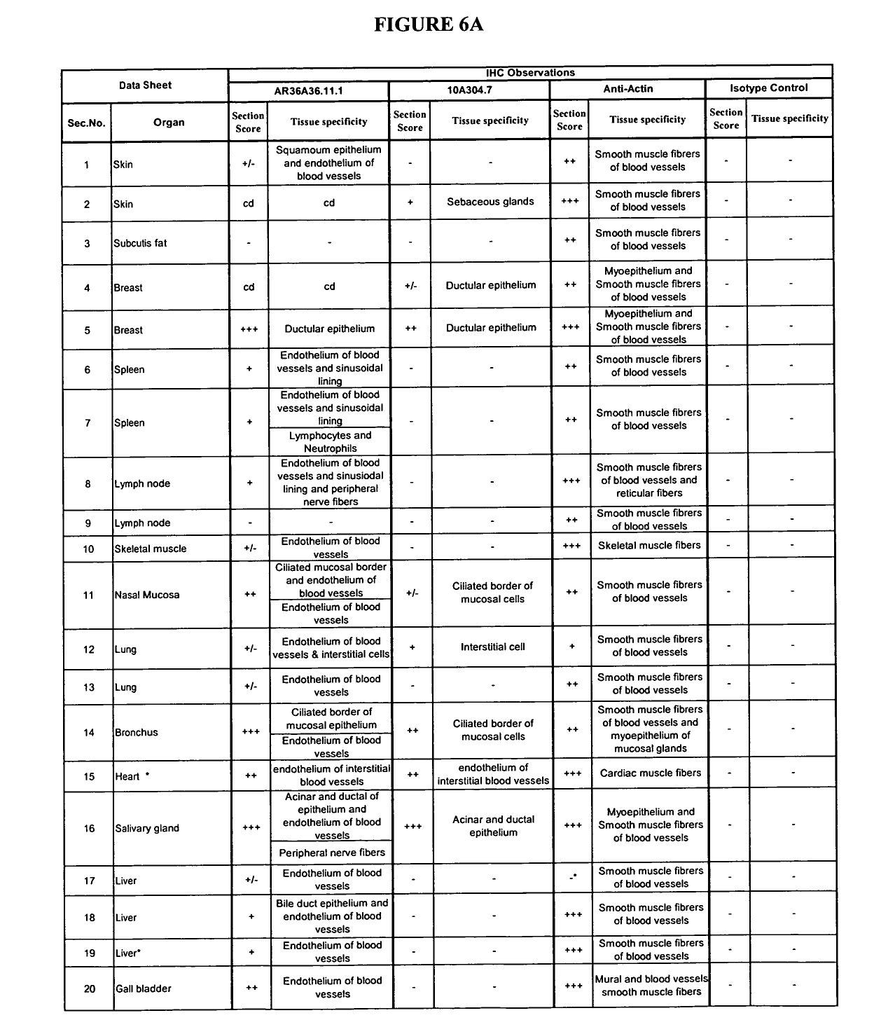

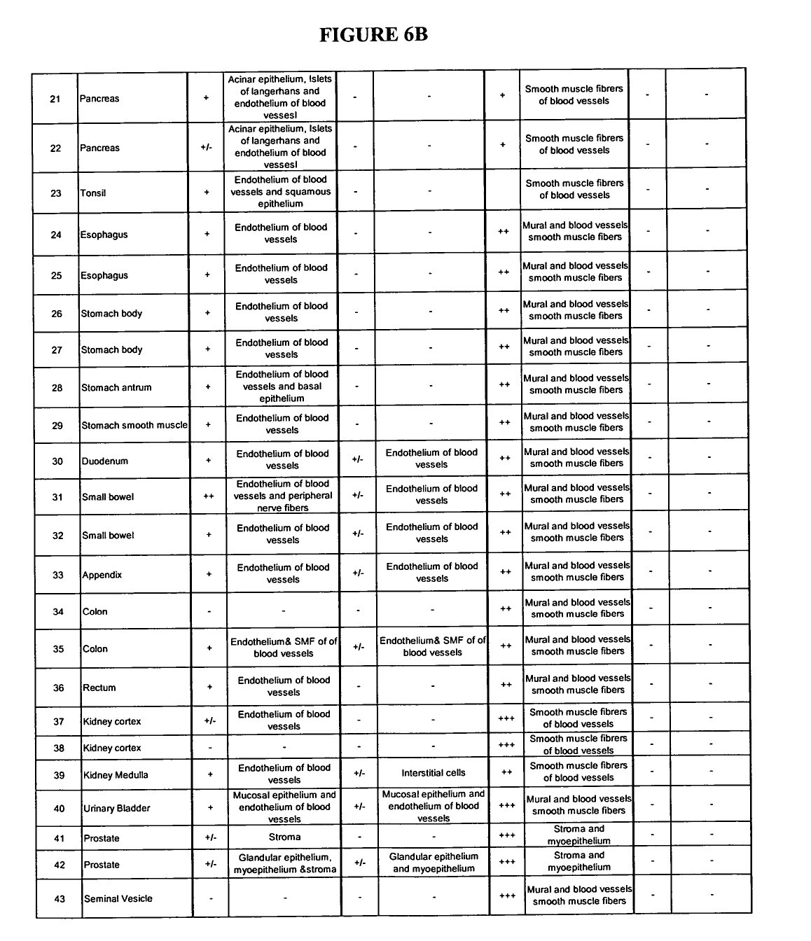

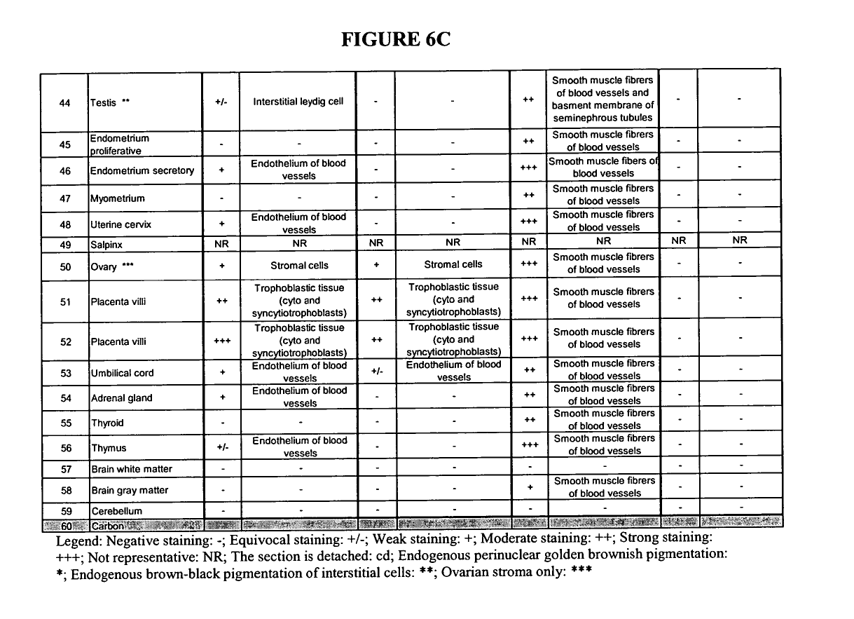

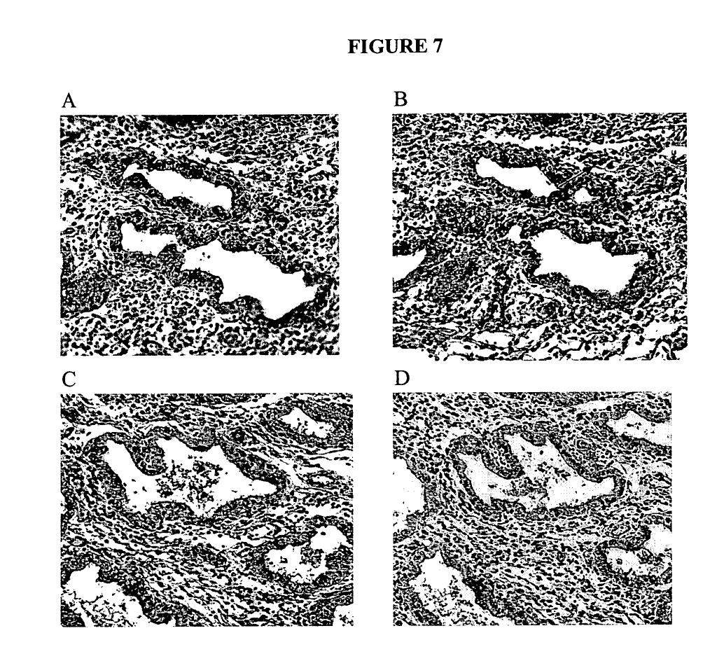

Binding of antibodies to 59 normal human tissues was performed using a human, normal organ tissue array (Imgenex, San Diego, Calif.). FIG. 6 presents a summary of the results of 10A304.7 and AR36A36.11.1 staining of an array of normal human tissues. The AR36A36.11.1 antibody bound predominantly to epithelial tissues (endothelium of blood vessels of various organs, squamous epithelium of skin and tonsils, ductular epithelium of breast, nasal mucosal epithelium, acinar and ductal epithelium of salivary glands, bile duct epithelium of liver, acinar epithelium and Islet of Langerhans of pancreas, mucosal epithelium of urinary bladder and glandular epithelium of prostate). The 10A304.7 antibody showed binding to spleenic lymphocytes and neutrophils, peripheral nerve fibers, smooth muscle fibers of blood vessels, interstitial (Leydig) cells of testis and trophoblastic tissue of placenta. The cellular localization was cytoplasmic and membranous with a diffuse staining pattern. The antibody bound predominantly to epithelial tissues (sebaceous glands of the skin, breast ductal epithelium, nasal mucosa, acinar and ductal epithelium of salivary glands, endothelium of blood vessels, mucosal epithelium of urinary bladder and glandular and myoepithelium of prostate). The antibody also showed binding to smooth muscle fibers and trophoblastic placental tissues. 10A304.7 bound to a subset of the human normal tissues that showed binding with AR36A36.11.1 (FIG. 7). The 10A304.7 and AR36A36.11.1 antibody have demonstrated binding to human tissue that is consistent with that previously reported for anti-CD59 antibodies. Therefore, the 10A304.7 and AR36A36.11.1 antibody are applicable for use in man.

### EXAMPLE 6

Human Tumor Tissue Staining

To determine whether the 10A304.7 or 36A36.11.1 antigen is expressed on human tumor tissues, the antibodies were individually tested on a multiple human tumor tissue array (Imgenex, San Diego, Calif.). The following information was provided for each patient: age, sex, organ and diagnosis. The staining procedure used was the same as the one disclosed in Example 5. The same positive and negative control antibodies were used as described for the human normal tissue array. All antibodies were used at a working concentration of 5 micrograms/mL.

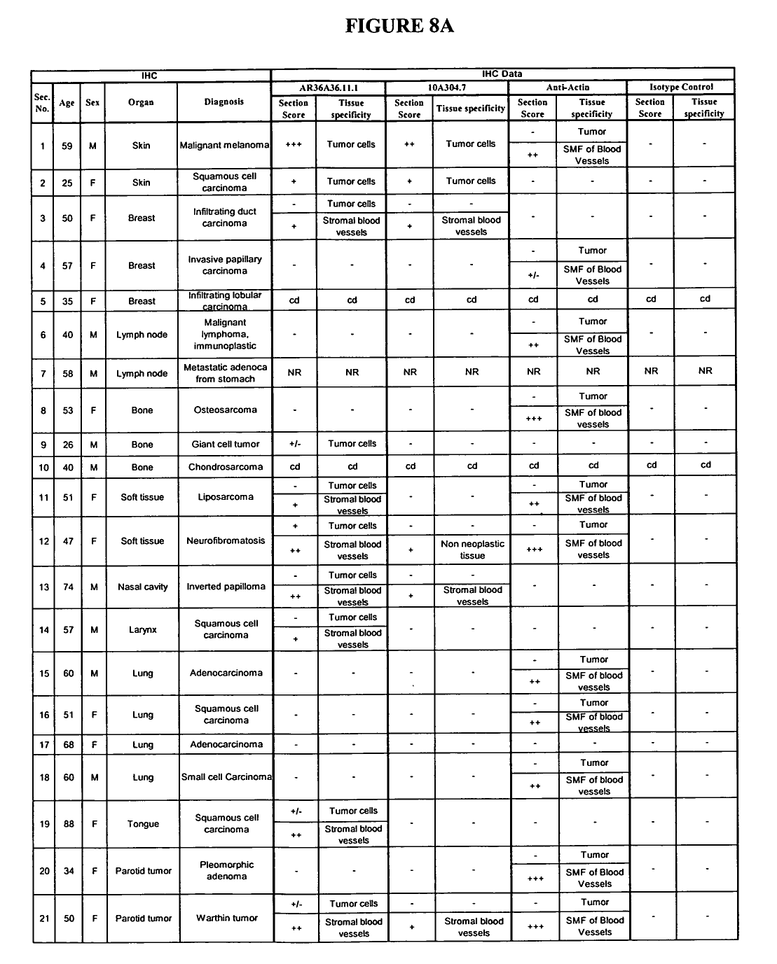

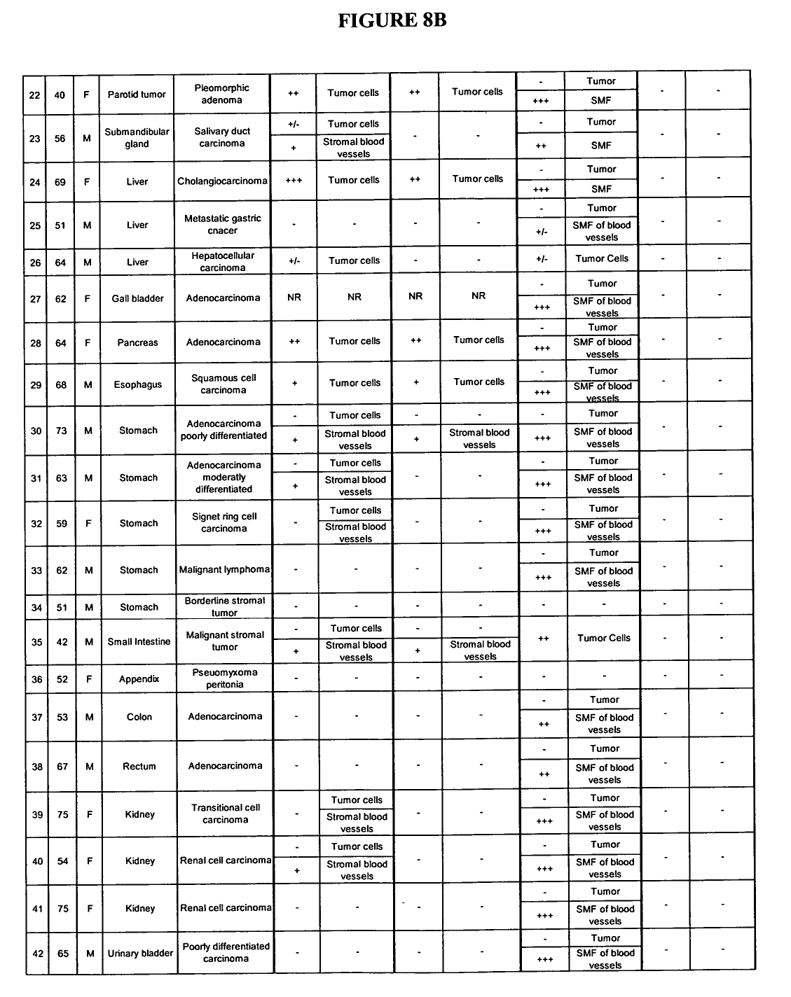

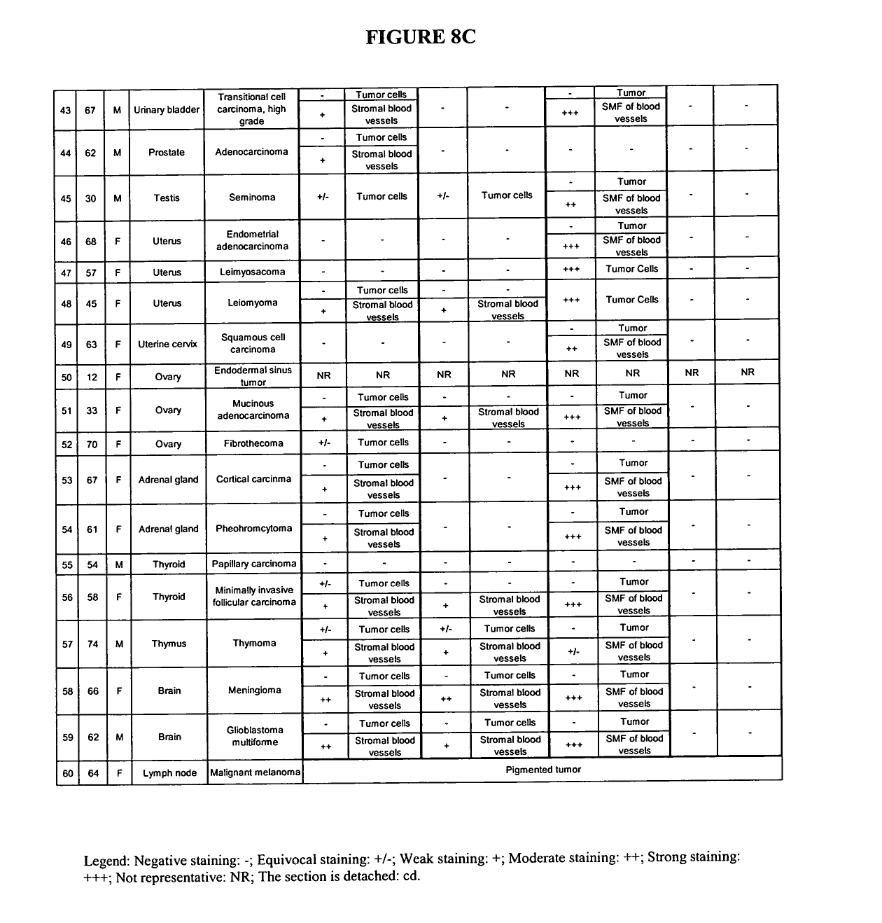

As disclosed in FIG. 8, the AR36A36.11.1 antibody bound to 17/54 (32%) of tested tumors. The antibody bound strongly to 2/17 tumors, moderately to 2/17, weakly to 4/17 and equivocally to 9/17. The tissue specificity was for tumor cells and stromal blood vessels. Cellular localization was membranous cytoplasmic with diffuse staining pattern. The 10A304.7 antibody bound to 9/54 (17%) of tested tumors. The antibody bound moderately to 4/54, weakly to 2/54, equivocally to 3/54 and there was no strong binding to any of the tested tumors. The tissue specificity was for tumor cells and stromal blood vessels. Cellular localization was membranous cytoplasmic with diffuse staining pattern. As with the normal human tissues, the 10A304.7 antibody bound to a subset of the tumors that AR36A36.11.1 bound to.

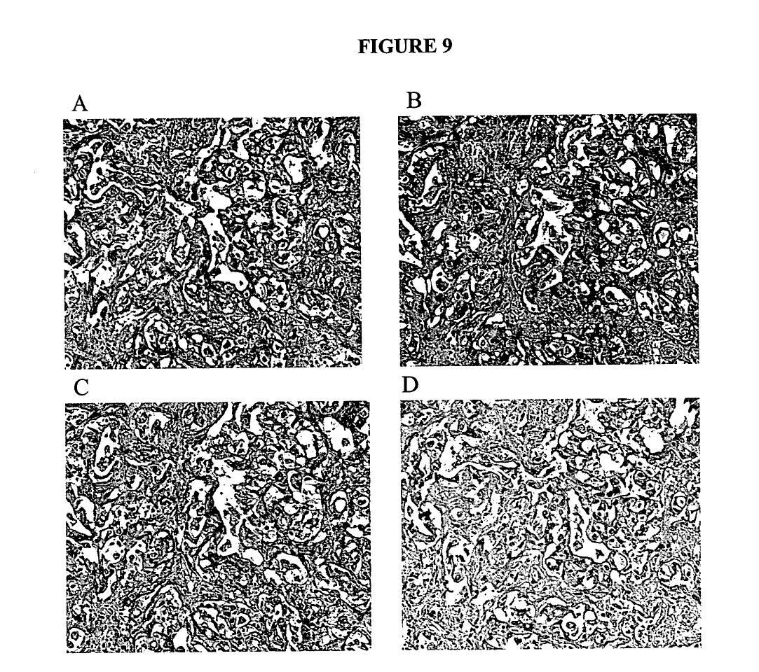

Therefore, it has been demonstrated that the 10A304.7 and AR36A36.11.1 antigen is located on the membranes of a variety of tumor types. These results indicate that 10A304.7 and AR36A36.11.1 antibodies have potential as therapeutic drugs in a wide variety of cancers including but not limited to cancers of the skin, liver (FIG. 9) and pancreas.

### EXAMPLE 7

Human Liver Tumor Tissue Staining

To further evaluate the binding of 10A304.7 to human liver tumor tissues, the antibody was tested on a liver tumor tissue array (Imgenex, San Diego, Calif.). The following information was provided for each patient: age, sex, organ and diagnosis. The staining procedure used was the same as the one disclosed in Example 5. The same negative control antibody was used as described for the human normal tissue array. The positive control antibody used was anti-AFP (alpha 1 fetoprotein; clone AFP-11 Abcam, Cambridge, Mass.). All antibodies were used at a working concentration of 5 micrograms/mL except for anti-AFP which was used at a working concentration of 10 micrograms/mL.

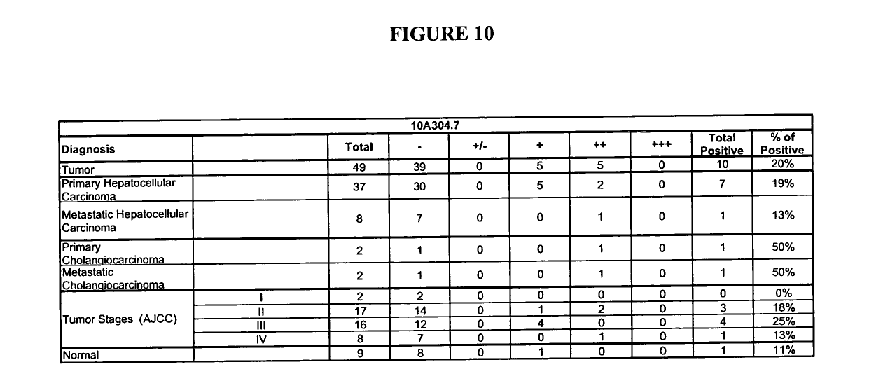

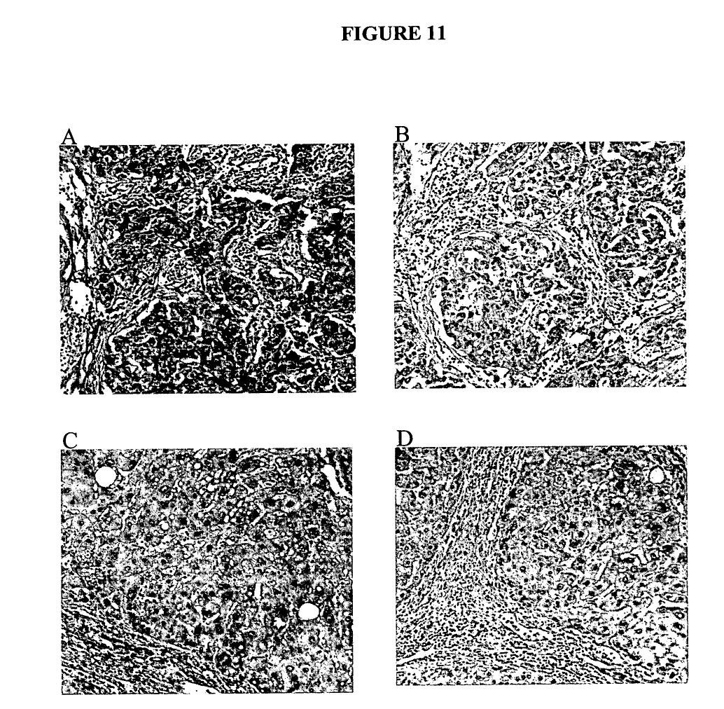

As disclosed in FIG. 10, 10A304.7 showed positive binding to 10/49 (20%) liver cancer sections with predominance in binding to primary hepatocellular carcinoma. Both primary and metastatic cholangiocarcinomas showed 50% binding with the antibody. The tissue specificity was to tumor cells and the endothelium of blood vessels. There was no relation between the binding of the antibody and the tumor stages. The antibody showed weak binding to 1/9 non-neoplastic liver tissue sections with restriction to the endothelium of small blood vessels (FIG. 11). The 10A304.7 antigen appears to be specifically expressed on liver tumor tissue. 10A304.7 therefore has potential as a therapeutic drug in the treatment of liver cancer.

The preponderance of evidence shows that 10A304.7 and AR36A36.11.1 mediate anti-cancer effects through ligation of epitopes present on CD59. It has been shown, in Examples 2 to 4, the 10A304.7 and AR36A36.11.1 antibody can be used to immunoprecipitate the cognate antigen from expressing cells such as MDA-MB-231 cells. Further it could be shown that the 10A304.7 and AR36A36.11.1 antibody could be used in detection of cells and/or tissues which express a CD59 antigenic moiety which specifically binds thereto, utilizing techniques illustrated by, but not limited to FACS, cell ELISA or IHC.

Thus, it could be shown that the immunoprecipitated 10A304.7 and AR36A36.11.1 antigen can inhibit the binding of either antibody to such cells or tissues using FACS, cell ELISA or IHC assays. Further, as with the 10A304.7 and AR36A36.11.1 antibody, other anti-CD59 antibodies could be used to immunoprecipitate and isolate other forms of the CD59 antigen, and the antigen can also be used to inhibit the binding of those antibodies to the cells or tissues that express the antigen using the same types of assays.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement of parts herein described and shown. It will be apparent to those skilled in the art that various changes may be made without departing from the scope of the invention and the invention is not to be considered limited to what is shown and described in the specification. One skilled in the art will readily appreciate that the present invention is well adapted to carry out the objects and obtain the ends and advantages mentioned, as well as those inherent therein. Any oligonucleotides, peptides, polypeptides, biologically related compounds, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A monoclonal antibody or ligand capable of specific binding to human CD59, in which said monoclonal antibody or ligand thereof reacts with the same epitope or epitopes of human CD59 as the isolated monoclonal antibody obtainable from hybridoma cell line 10A304.7 having ATCC Accession No. PTA-5065; said monoclonal antibody or ligand being characterized by an ability to competitively inhibit binding of said isolated monoclonal antibody to its target human CD59 antigen.

2. A monoclonal antibody or ligand capable of specific binding to human CD59, in which said monoclonal antibody or ligand thereof reacts with the same epitope or epitopes of human CD59 as the isolated monoclonal antibody obtainable from hybridoma cell line AR36A36.11.1 having IDAC Accession No. 280104-02; said monoclonal antibody or ligand being characterized by an ability to competitively inhibit binding of said isolated monoclonal antibody to its target human CD59 antigen.

3. A monoclonal antibody or ligand that recognizes the same epitope or epitopes as those recognized by the isolated monoclonal antibody produced by a hybridoma selected from the group consisting of hybridoma cell line 10A304.7 having ATCC Accession No. PTA-5065 and hybridoma cell line AR36A36.11.1 having. IDAC Accession No. 280104-02; said monoclonal antibody or ligand being characterized by an ability to competitively inhibit binding of said isolated monoclonal antibody to its target human CD59 antigen.

4. A process for treating a human cancerous tumor which expresses human CD59 antigen comprising:
administering to an individual suffering from said human cancer, at least one monoclonal antibody or ligand that recognizes the same epitope or epitopes as those recognized by the isolated monoclonal antibody produced by a hybridoma selected from the group consisting of hybridoma cell line 10A304.7 having ATCC Accession No. PTA-5065 and hybridoma cell line AR36A36.11.1 having IDAC Accession No. 280104-02;
wherein binding of said epitope or epitopes is effective in reducing tumor burden.

5. A process for treating a human cancerous tumor which expresses human CD59 antigen comprising:
administering to an individual suffering from said human cancer, at least one monoclonal antibody or ligand that recognizes the same epitope or epitopes as those recognized by the isolated monoclonal antibody produced by a hybridoma selected from the group consisting of hybridoma cell line 10A304.7 having ATCC Accession No. PTA-5065 and hybridoma cell line AR36A36.11.1 having IDAC Accession No. 280104-02; in conjunction with at least one chemotherapeutic agent;
wherein said administration is effective in reducing tumor burden.

6. A binding assay to determine a presence of cancerous cells which express an epitope or epitopes of CD59 in a tissue sample selected from a human tumor comprising:
providing a tissue sample from said human tumor;
providing at least one monoclonal antibody or ligand that recognizes the same epitope or epitopes as those recognized by the isolated monoclonal antibody produced by a hybridoma selected from the group consisting of hybridoma cell line 10A304.7 having ATCC Accession No. PTA-5065 and hybridoma cell line AR36A36.11.1 having IDAC Accession No. 280104-02;
contacting said at least one monoclonal antibody or ligand thereof with said tissue sample; and
determining binding of said at least one monoclonal antibody or ligand thereof with said tissue sample;
whereby the presence of said cancerous cells in said tissue sample is indicated.
